(19) United States
(12) Patent Application Publication
(10) Pub. No.: US 2004/0198650 A1
(43) Pub. Date: Oct. 7, 2004

(54) Means for inhibiting proteolytical processing of parkin

(76) Inventor: Poul Jensen, Hojbjerg (DK)

Correspondence Address: BROWDY AND NEIMARK, P.L.L.C., 624 NINTH STREET, NW, SUITE 300, WASHINGTON, DC, 20001-5303, US

(21) Appl. No.: 10/473,226

(22) PCT Filed: Apr. 2, 2002
(86) PCT No.: PCT/DK02/00221

Related U.S. Application Data
(60) Provisional application No. 60/281,286, filed on Apr. 3, 2001.

(30) Foreign Application Priority Data
Mar. 29, 2001 (DK) PA 2001 00525

(51) Int. Cl. A61K 38/17; C12Q 1/68; C07K 14/705

(52) U.S. Cl. 514/12; 435/6; 435/69.1; 435/320.1; 435/325; 530/350; 536/23.5

(57) ABSTRACT

An isolated nucleic acid sequence encoding a polypeptide functionally homologous to the wild type form of the Parkin polypeptide or a fragment of said polypeptide, the amino acid sequence of said polypeptide differing from the wild type amino acid sequence at least in including a mutation at Asp 126, wherein the said nucleic acid sequence is at least 70% identical to a nucleic acid sequence encoding the wild type Parkin polypeptide, and a mutated Parkin polypeptide.

### BACKGROUND OF THE INVENTION

The symptomatology of parkinsonism comprises bradykinesia, tremor, postural instability and rigidity and is caused by a loss of dopamine producing neuronal cells in the pars compacta of the substantia nigra (SN). Parkinson's disease (PD) is the most common cause of parkinsonism but other diseases e.g. autosomal recessive juvenile parkinsonism (AR-JP) can cause the symptoms as well.

Parkinson's disease (PD) is a neurodegenerative disorder characterised by the appearance of intracytoplasmic inclusions, so called Lewy bodies (LB), in dopaminergic neurones in the substantia nigra and the progressive loss of these neurones.

The degenerative nerve cell phenotype of Parkinson's disease is shared by other neurodegenerative disorders, such as e.g. Alzheimer's disease (AD) and Huntington's disease (HD), where inclusions develop that contain filaments of tau protein or Huntingtin fragments, respectively. Recently it has been shown in a transgenic mice model that a continues influx of the mutant Huntingtin protein is required to maintain these symptoms, raising the interresting possibility that at least HD may be reversible (Yamamoto et al., (2000) Cell 101:57-66).

During development of the nervous system, cell death is a normal feature. Elimination of substantial numbers of initially generated cells enables useful pruning of “mismatched” or excessive cells produced by exuberance during the proliferative and migratory phases of development. Such cell death, occurring by “programmed” pathways, is termed apoptosis. In mature organisms, cells die in two major fashions, either by necrosis or apoptosis. In the adult nervous system, because there is little cell production during adulthood, there is little normal cell death. However, neurological diseases are often associated with significant neural cell death, particularly in response to various physiological stresses, such as hypoxia or ischemia.

There are a number of morphological changes shared by cells undergoing apoptosis, including plasma and nuclear membrane blebbing, cell shrinkage (condensation of nucleoplasm and cytoplasm), organelle relocalisation and compaction, chromatin condensation and production of apoptotic bodies (membrane enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall (1981), pp. 9-34), wherein a cell activates an internally encoded suicide program that results in apoptotic cells and bodies which are usually recognised and cleared by neighbouring cells or macrophages before lysis. Because of this clearance mechanism, inflammation is not induced despite the clearance of great numbers of cells (Orrenius, S., J. Internal Medicine 237:529-536 (1995)). Necrosis on the other hand is a much less controlled mode of cell death that ultimately leads to cell leakage and inflammatory responses.

Acute disorders, occurring over minutes to hours, such as brain trauma, infarction, haemorrhage, or infection, prominently involve cell death, much of which is executed through necrosis. Whereas chronic disorders, with relatively slow central nervous system degeneration, may occur over years or decades and involve a significantly slower process of cell loss. There is evidence that the mechanism of neuronal cell death in these chronic disorders involve apoptosis. Direct conclusive evidence of apoptosis in these chronic disorders is scarce, though, because of the swiftness of cell death in relation to the slowness of the disease. Thus, at any particular time point of assessment, very few cells are actually undergoing death.

Of significance is though, that while treating the underlying causes of these conditions is an admirable goal, it may also be possible to develop productive therapies based on alleviating the process of cell death. This is particularly likely if the cell loss is executed through apoptosis, for which the molecular cascade is increasingly understood.

Apoptosis is an active process and thus dependent on ATP. It is a complex scenario orchestrated by cell surface receptors (e.g., CD95/APO-1/Fas; TNF receptor) and their ligands (CD95-L; TNF) as well as evolutionarily conserved mechanisms, including mitochondrial factors (e.g., Bcl-2-related proteins, reactive oxygen species, mitochondrial membrane potential, opening of the permeability transition pore) or p53. The actual proteolytic cascade involves the sequential activation of a family of cysteine proteinases, the caspases, which are modified by several endogenous activators and inhibitors. Interestingly, recent studies have shown that activated caspases are present in affected cells of neurodegenerative diseases (Cervais et al., (1999) Cell 97:395-406) and a neurodegenerative phenotype being neither apoptotic nor necrotic has been demonstrated in Huntington disease (Turmaine et al., (2000) Proc Natl Acad Sci USA, 97:80938097). This indicates that a prolonged “low level” pro-apoptotic state exists that is kept at bay by cytoprotective agents.

In PD patients, increased numbers of apoptotic neuronal nuclei have recently been identified in melanized neurones of the substantia Nigra and increased immunoreactivity against caspase 3 and Bax could also be shown. Furthermore, GAPDH nuclear accumulation was observed in the substantia nigra of these patients, suggesting apoptotic rather than necrotic cell death (Tatton, N A, Exp Neurol (2000) Nov; 166(1):29-34).

The cause of the selective degeneration of nigrostriatal neurones in Parkinson disease (PD) has remained largely unknown. Exceptions hereto include rare missense mutations in the alpha-synuclein gene on chromosome 4, a potentially pathogenic mutation affecting the ubiquitin pathway, and mutations in the parkin gene on chromosome 6. However, the latter mutation is not associated with the formation of typical Lewy bodies. In classical PD, though, a biochemical defect of complex I of the mitochondrial respiratory chain has been described in a relatively large group of confirmed cases. Recent cybrid studies indicate that the complex I defect in PD has a genetic cause and that it may arise from mutations in the mitochondrial DNA. Sequence analysis of the mitochondrial genome supports the view that mitochondrial point mutations are involved in PD pathogenesis. However, the exact involvement of mitochondrial function in PD remains unresolved (Kosel et al., Biol Chem 1999 Jul-Aug;380(7-8):865-70).

Genetic studies have further shown that the parkin gene codes for a 52 kDa protein that is vital for the survival of dopamine producing neurones of the substantia nigra (Kitada et al., (2000) Nature 392:605-608). Parkin gene lesions are a common causes for early on-set of Parkinson's disease in AR-JP(Locking et al., (2000) N Engl. J Med 342:1560-1567).

Parkin is likely to function in the cellular catabolism of specific proteins as it exhibits ubiquitin ligase activity in vivo and in vitro, and displays specificity towards both specific ubiquitin conjugases and cellular targets for ubiquitination (Shimura et al., (2000) Nat Genet 25:302-305; Imai et al., (2000) J Biol Chem Vol.275, No.46, Nov 17, pp. 35661-35664). It's cytoprotective role is likely to rely on a protection against specific noxious stimuli from e.g. the unfolded protein response (Imai et al., (2000) J Biol Chem Vol.275, No.46, Nov 17, pp. 35661-35664). Factors that inhibit Parkin function may thus contribute to the progression of Parkinson's disease and could be candidate targets for therapeutic interventions. Parkin may also inhibit a certain type of cell death through proteasome-mediated protein degradation. In this sense, accumulation of misfolded proteins in the endoplasmatic reticulum (ER) would constitute an unfolded protein stress, which could lead to cell death.

Recently, immunohistochemical stainings of brain tissue sections revealed that melanin-containing pigmented neurones in the substantia nigra (SN) in PD patients and in control subjects contain Parkin, whereas the same neuronal population in AR-JP patients did not show any Parkin protein staining at all. Nonetheless, Parkin protein in the SN of PD patients was found to be reduced, a finding that is in agreement with the loss of nigral neurones in PD patients and even more intriguingly, a second, processed form of Parkin was found in these patients. The inhibition of Parkin function can thus be due to reversible or irreversible ligand interactions or post translational modifications as indicated by the presence of a 41 kDa putative proteolytic fragment of Parkin in brain extracts from Parkinson's disease patients (Shimura et al., (1999) Ann Neurol 45:668-672).

In conclusion, while the underlying causes of PD are.still to be investigated, an alternative possibility is to develop productive therapies based on alleviating the process of cell death by e.g. preventing the proteolytic cleavage of Parkin in SN neurones. Furthermore, if PD is a reversible process, as postulated for HD, and it's symptomatic progression depends on the availability of cleaved Parkin, or the absence of functional and/or uncleaved Parkin, preventing the cleavage of Parkin will even open up new means of successfully treating and/or preventing PD.

### OBJECT OF INVENTION

The object of the invention is related to preventing and/or altering proteolytic cleavage of Parkin for analysis, prophylactics and treatment of a human and/or mammal with PD and potentially any other neurological disorder that is associated with caspase-associated cell death or apoptotic cell loss.

### SUMMARY OF INVENTION

The present invention comprises the means for analysis, prophylactics and treatment of a mammal with Parkinson's disease.

The invention relates to the surprising finding that the Parkin protein harbours a potential cleavage site for cysteine proteases, such as caspases, and that it is proteolytically processed during apoptosis.

The inventors further prove that Parkin is actually at least partially cleaved by caspase mediated proteolysis during apoptosis and that an alteration of a potential cleavage site at Asp126 prevents said cleavage. As the cleavage of Parkin abrogates the cytoprotective function of Parkin in the cell, the inhibition of Parkin cleavage at Asp 126 or in the close vicinity thereof, represents a novel and exciting possibility to alter or even abrogate the successive loss of Parkin in PD and thereby halting or delaying the loss of neurones in PD patients. The present invention therefor relates to different methods for inhibiting Parkin cleavage at said specific cleavage site at Asp126.

Activation of caspases in human neurones does not lead to an immediate and rapid process of cell death but provokes a protracted form of apoptosis. Activation of caspases in human neurones may thus participate in the long-term cleavage of Parkin and other potential toxic fragments resulting from caspase-mediated proteolysis.

The present invention comprises an isolated nucleic acid sequence that encodes a mutated Parkin protein, that is no longer cleavable by cysteine proteases at its amino acid sequence at position number 126. Said mutation can either be a substitution of the wild type Asp 126 with any other amino acid such as Ala, Cys, Glu, Phe, Gly, His, lle, Lys, Leu, Met, Asn, Pro, Gin, Arg, Ser, Thr, Val, Trp and Tyr, or a mere deletion of said Asp 126. A preferred embodiment of the invention is shown in SEQ ID NO. 3, a nucleic acid sequence coding for a protein with an amino acid sequence as shown in SEQ ID NO.4, wherein aspargine at amino acid residue number 126 is exchanged with glutamate.

Said nucleic acid sequence or a fragment of it that at least comprises the amino acid position 126, can be used for preventing or treating PD in a patient in a multitude of ways, or for detecting and/or diagnosing a potential disposition of a subject for the disease, but can as well be used for treating, preventing or diagnosing other neurological disorders that are associated with neuronal loss due to caspase-associated cell death and/or apoptosis.

Said nucleic acid furthermore codes for a protein that is at least 70% identical to Parkin and that has at least a deletion at Asp 126 or a substitution of Asp 126 for another amino acid. Said protein is thus not cleavable or proteolytically processable by a cysteine protease at this site, such as a protease belonging to the family of caspases.

A protein or a fragment of a protein, as described in the present invention, comprises at least said deletion or substitution at wild type Asp126 and can be of use for numerous ways of treating, preventing and/or diagnosing PD. E.g., said protein or fragment of a protein can be used to develop an immunogenic substance, such as a monoclonal or polyclonal antibody that selective recognises uncleaved wild type Parkin or mutated Parkin.

In the scope of the present invention is also the surprising insight that proteolytical processing of Parkin at its potential cleavage site Asp 126, is an indicator for the severity of neurological stress, on-set of PD and/or the potential disposition of a subject to develop PD. Like many other chronical neurological diseases that are associated with neuronal loss, PD develops during a long period of time and can be influenced by a vast amount of different neurological stresses. The neurological stresses accumulate over a period of months or years, leading to increased cell loss in the CNS of a subject. An important player in the accumulation and the decreased tolerance for neurological stress is cleavage of Parkin by proteases. Uncleaved Parkin is essential for the neurone as it protects it against unfolded protein stress, thus the absence of uncleaved Parkin and/or the presence of Parkin cleaved at Asp126 is an excellent indicator for the severity of a neurological disorder that is associated with neuronal apoptosis. In the same way, absence of uncleaved Parkin and/or presence of Parkin cleaved at Asp126 can be analysed to determine whether a subject is prone to develop a neurological disorder associated with neuronal cell loss, such as for example PD.

Furthermore, as absence of uncleaved Parkin and/or the presence of Parkin cleaved at Asp126 is an excellent indicator for the severity of a neurological disorder, this tool can of course be used to measure how well a patient is responding to a given treatment and/or to evaluate the right dosage of a medication in each single case or in specific groups of individuals. The detection of proteolytical processing of Parkin can be detected in any common sample from a patient or in cell cultures and/or primary neuronal cultures grown from samples taken from said patient. Another embodiment of the present invention also envisions the use of detecting said Parkin processing for evaluating the effectivity of any pharmaceutical substance in vitro, such as in cell-cultures, primary cell systems and/or cell-free systems.

Caspases have prior to the present invention been reported to be able to release an aminoterminal fragment comprising the first 86 or 87 amino acid residues of the protein (Tsai et al., 2000). In addition hereto, the finding of the present invention directly demonstrates that caspase activated activity cleaves Parkin during apoptosis and that said cleavage is performed at Asp126 or directly before or after this amino acid residue, as seen in SEQ ID No: 2. Moreover, Asp 86 and/or Asp 87 do not represent a significant cleavage site during apoptosis, as determined by site-directed mutagenesis (see example 6). An especially preferred embodiment of the present invention thus relates to the prevention of Parkin cleavage by cysteine proteases, such as belonging to the family of caspases, for treating or preventing PD.

The present invention relates to novel methods for the preservation of Parkin function in nervous tissue. In one embodiment, Parkin function is preserved by exposing the protein in the tissue to substances or small organic compounds that inhibit proteolysis of Parkin. As the proteolysis of Parkin is often disease-associated, it is envisioned that the prevention of Parkin cleavage will either prevent and/or delay disease-associated cell-death in nervous tissue. Said treatment will be neuroprotective in Parkinson's disease and other disorders where Parkin dysmetabolism contributes to the disease progression.

In the present context, such substances and/or organic compounds are comprised in the group consisting of peptide and non-peptide inhibitors of caspases and caspase activators and peptide and non-peptide ligands for Parkin that prevent the cleavage of Parkin after Asp 126.

Small organic compounds (SOC) and peptides comprised by the current invention are organic molecules that modulate the cleavage of parkin after D126.

The present invention further relates to the preservation of Parkin function, wherein said preservation relies on the inhibition or stimulation of signalling pathways that impinge on Parkin. In this particular embodiment, Parkin is rendered less prone to proteolytic cleavage. The present invention thus relates to methods that preserve Parkin from proteolytic cleavage by cysteine proteases, such as e.g. caspases and/or other proteases that can be activated by caspases. In this context, the tissue can be treated with protease inhibitors, phosphatase inhibitors, phosphatase activators, kinase inhibitors and/or kinase activators.

In an especially preferred embodiment, neuronal tissue is exposed to the nucleic acids or proteins of the present invention, restoring the presence of uncleaved Parkin in neuronal cells either by implanting or injecting the mutant protein or a vector that will express the mutant protein and/or transplanting transformed neuronal cells, such as for example stem cells that express mutated Parkin, according to the present invention and thus uncleavable by caspase mediated proteolysis, into the CNS. In this embodiment, the development of PD can either be altered, postponed and/or suppressed by the presence of uncleaved Parkin in the cells. In an even more preferred embodiment, the disease is reversed by the presence of uncleaved Parkin in the cells and thus the patient is healed.

### LEGENDS TO FIGURES

FIG. 1:

The amino acid sequence of human Parkin is deduced from the cDNA nucleotide deposited in Genbank by the accession number AB009973. The arrow indicates the position of Asp126. Mutation of this amino acid residue abrogates the apoptosis-associated cleavage of Parkin. The sequence is shown in single letter code.

FIG. 2:

Panel A: Localisation of the epitopes used for producing the antibodies PAR-N1, PAR-C1 and T160.

Panel B: The Chinese hamster ovary (CHO) cell line K1 (lane 1) and the human neuroblastoma cell line SH-SY5Y (lane 5) were stably transfected with an expression vector that lead to expression of human Parkin protein in the cell lines CH0-K3 (lanes 2-4) and SHSY5Y-Park 8 (lane 6). The immunoblot was probed with T160 (lanes 1, 2, 5, 6) PAR-N1 (lane 3) and PAR-C1 (lane 4).

Panel C: Immunoprecipitation of metabolically labelled CHO-K3 cells expressing recombinant human Parkin. The extract from the metabolically labeled cells were immunoprecipitated with PAR-N1 (lane 1), PAR-C1 (lane 2), T160 (lane 3) and non-immune serum (lane 4). The arrow indicates the position of the 52 kDa parkin protein.

FIG. 3:

Parkin cleavage during cellular apoptosis. The stably transfected Parkin expressing cell lines, K3CHO (lanes 1-5) and Park8 SH-SY5Y (lanes 6, 7) were cultured in the absence (lanes 1) and presence of the inducers of apoptosis; okadaic acid (200 nM for 16 hours, lanes 2, 5-7), staurosporin (10 microMwml for 16 hours, lane 3) and campthotecin (5 microgram/ml for 30 hours, lane 4) whereafter the cell extracts were resolved by SDS-PAGE and immunoblotting. Lanes 1-4, 6 were probed with T160, lane 5 with PAR-N1 and lane 7 with PAR-C1. The molecular weight is indicated to the left for Parkin (51 kDa) and the Parkin peptides (38 kDa and 11,5 kDa, respectively). The induction of apoptosis was verified biochemically by testing the binding of the same membranes with the mouse monoclonal poly-ADP-ribose (PARP) antibody 66401 A (Pharmigen). This antibody recognised only the full length 116 kDa PARP protein in the control cells whereas the caspase 3 generated 86 kDa fragment was generated in the cells treated with the inducers of apoptosis. The generation of the 86 kDa PARP fragment is a recognised marker for apoptosis associated proteolysis. The absent binding of PAR-N1 to the 38 kDa band, as compared to PAR-C1 and T160 indicates the proteolysis liberates a approximately 12 kDa N-terminal band from the larger C-terminal fragment. The N-terminal fragment is recognised by the T160 antibody in lanes 2 and 6.

FIG. 4:

Caspase inhibitors inhibit the apoptosis-associated Parkin cleavage. K3 CHO cells were treated with 200 nM okadaic acid for 16 h (lanes 2-4) in the presence of 10 microM of the caspase inhibitors YVAD-chloromethylketone (lane 3) and DEVD-chloromethylketone (lane 4). The control cells treated with the DMSO used for solubilising the caspase inhibitors are shown in lane 2. Lane 1 represents non-treated control cells. The arrows to the left demonstrate the position of the 51 kDa Parkin and 38 kDa Parkin fragment. Evidently, the cleavage is abrogated by the inhibitors. Similar data was obtained in the Park8 SH-SY5Y cells.

FIG. 5:

Site directed mutagenesis of Asparagine 126 in parkin to a glutamate residue reduce the apoptosis associated cleavage of parkin. Caspases are proteinases that always cleave after an Asp residue and FIG. 3 indicated the cleavage takes place within the N-terminal 150 amino acid residues. We mutagenised the parkin expression vector to express human parkin where the Asp in the positions 86 plus 87,106, 115, 126 and 130 were substituted with and Glu. CHO and SHSY5Y cell lines were stably transfected with the expression vectors and used for testing the effect of the mutations on apoptosis-associated parkin cleavage. These cells and the cell lines expressing the normal Parkin were treated with 200 nanoM okadaic acid for 16 h and extracted for T160 immunoblotting. The CHO cell lines are represented in lanes 1-6 and the SH-SY5Y cell lines in lanes 7-12. Wild type Parkin is represented in lanes 1, 2, 7, 8; D126E in lanes 3, 4, 9, 10; D130E in lanes 5, 6, 11, 12. Lanes 1, 3, 5, 7, 9, 11 represent untreated cells and lanes 2, 4, 6, 8, 10, 12 represent okadaic treated cells. The positions of the 51 kDa and 38 kDa Parkin proteins are indicated by the upper and lower left arrows. Note the drastically reduced parkin cleavage in the D126E cells. The double mutant DD86, 87EE and the D105E and D115E cell lines demonstrated a cleavage as the WT parkin and the D130E cells. The significant inhibition of the apoptosis-associated cleavage upon mutation of D126 demonstrates that cleavage at or near this residue represent the major cellular apoptosis-associated cleavage site.

FIG. 6:

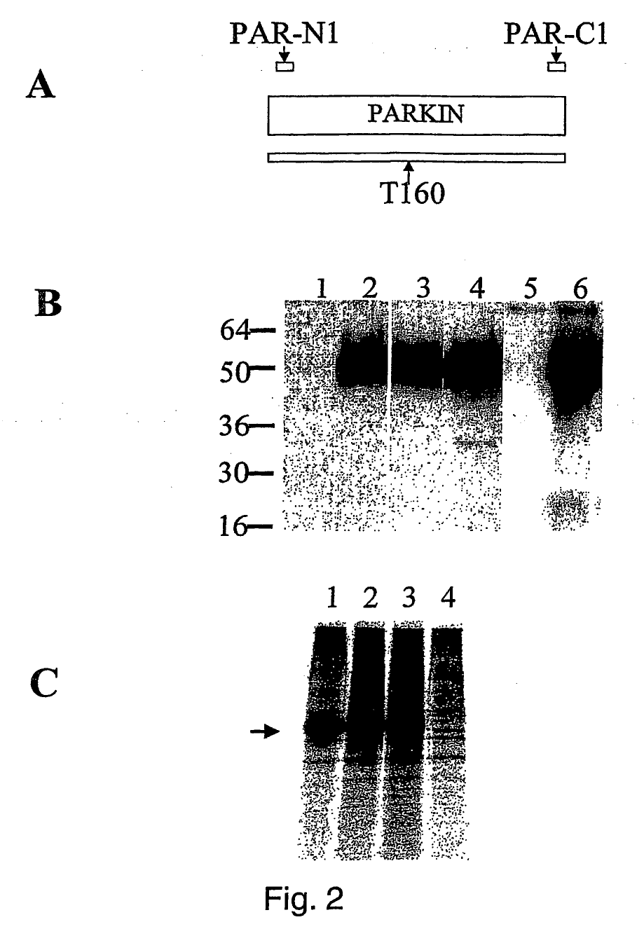

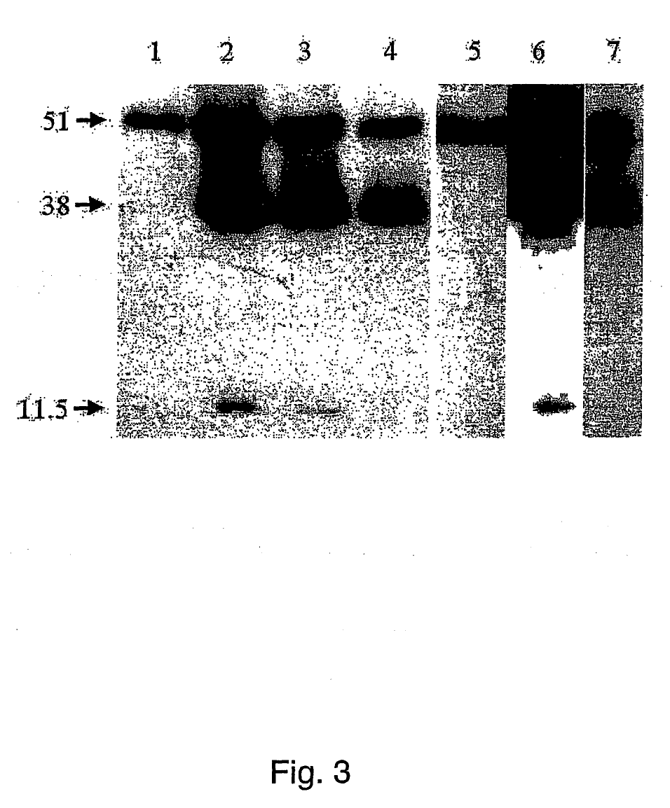

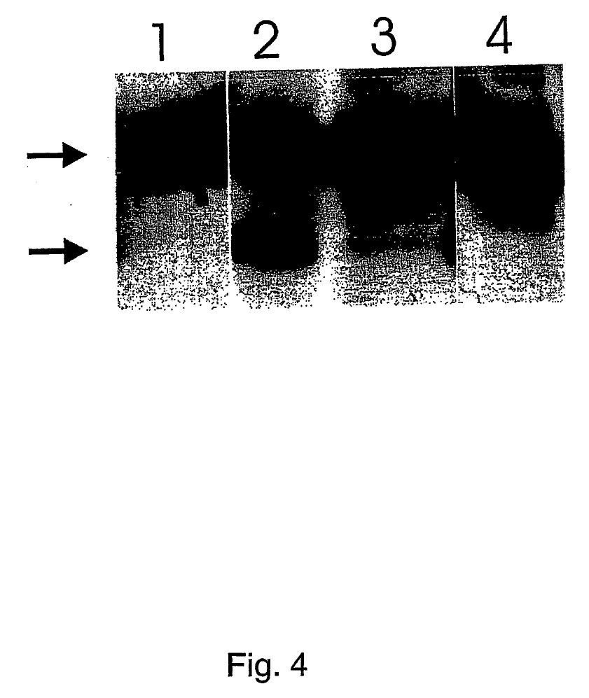

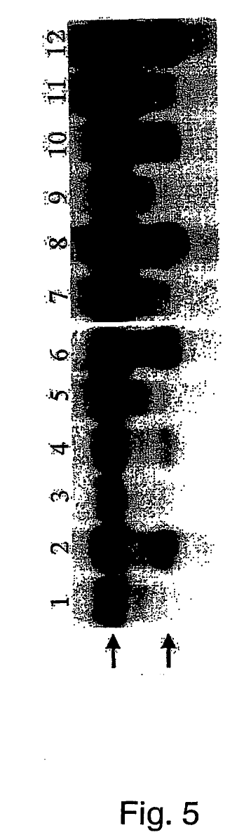

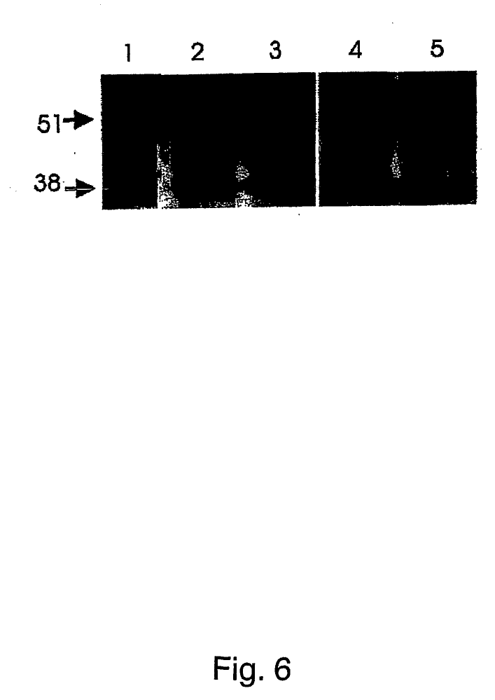

Detection of a 38 kDa parkin fragment in substantia nigra tissue from Parkinson's disease, but not age matched controls. Frozen substantia nigra tissue (1 gram) from patients dying with Parkinson's disease and agee matched controls with no known diseases In the central nervous system was extracted in 5 ml 0.32 M sucrose, 2 mM EDTA, 0.5 mM dithioerythreitol, 10 mM Tris, pH 7.4 supplemented with the Complete proteinase inhibitor cocktail from Roche Biochemicals. All procedures were performed on ice. The homogenates (100 microgram protein) were heated to 950C in 2% SDS, 20 mM dithioerythreitol, 20 mM Tris, pH 6.5, 20% glycerol, resolved by 10-20% gradient SDS-polyacrylamide gel electrophoresis, subjected to electroblotting and probed with the T160 antibody as described in FIG. 2. Lane 1 represents an extract from an okadaic acid treated CHO-K3 cell, lanes 2, 3 represents Parkinson's disease tissue and lanes 4, 5 represents age matched controls. The molecular size of the Parkin bands is indicated to the left. Note the distinct, although weak 38 kDa fragment in the Parkinson's disease tissue. The controls contain no distinct band but merely a smear covering a larger molecular size range.

### DETAILED DESCRIPTION

Cysteine proteases from the family of caspases have prior to the present invention been reported to be able to release an aminoterminal fragment comprising the first 86 or 87 amino acid residues of Parkin. In the present application, the inventors demonstrate that caspase activated activity, likely represented by an activated caspase itself, actually cleaves Parkin during cellular apoptosis, that this cleavage is performed close to Asp126 or directly before or after this amino acid residue (see SEQ ID NO: 2) and that this cleavage abrogates the cytoprotective function of Parkin by liberating the N-terminal target binding site from the C-terminal ubiquitin conjugase binding site.

Nucleic Acid Molecules of the Invention

The inventors further show that an alteration of a potential cleavage site at Asp126 prevents said cleavage. The present invention therefore comprises an isolated nucleic acid sequence that encodes a mutated Parkin protein, that is no longer cleavable by cysteine proteases at its amino acid sequence at position number 126 or in close vicinity thereof. Said mutation can either be a substitution of the wild type Asp 126 with any other amino acid such as Ala, Cys, Glu, Phe, Gly, His, lle, Lys, Leu, Met, Asn, Pro, Gin, Arg, Ser, Thr, Val, Trp and Tyr, and/or a deletion of said Asp 126.

In the present context, at “Asp 126” describes the peptide bond after Asp 126, which is C-terminal of this residue.

The isolated nucleic acid sequence of the present invention, or a fragment of said nucleic acid sequence is at least 70% identical to a nucleic acid sequence, such as shown in SEQ ID NO: 3, or to a fragment of SEQ ID NO: 3, respectively, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or at least 100% identical, and codes for a polypeptide functionally homologous to a mutated Parkin polypeptide or a fragment of said polypeptide, wherein the amino acid sequence of said polypeptide differs from the wild type amino acid sequence, as shown in SEQ ID NO: 2, at least in including a mutation at wild type Asp 126. A preferred embodiment of the invention is shown in SEQ ID NO. 3, coding for a protein with an amino acid sequence as shown in SEQ ID NO:4, wherein aspargine at residue number 126 is exchanged with glutamate, thereby altering and/or destroying the potential cleavage site of said mutated Parkin at D126. In the context of this application, said mutated Parkin is referred to as Parkin D126. Nucleotide 479 in SEQ ID NO: 3 can alternatively be changed to an adenine and still encode for the same amino acid sequence, shown in SEQ ID NO: 4, thus, such an alternative sequence is of course also encompassed in the scope of the present invention.

Sequence identity as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “Similarity” can readily be calculated by known methods.

By a polynucleotide having a nucleotide sequence at least, for example, 95% identical to a reference nucleotide sequence as shown in SEQ ID No.3, is intended that the nucleotide sequence of the polynucleotide is identical to the reference sequence except that the polynucleotide sequence may include up to 5 point mutations per each 100 nucleotides of the reference nucleotide sequence as shown in SEQ ID No.3. In other words, to obtain a polynucleotide having a nucleotide sequence at least 95% identical to a reference nucleotide sequence: up to 5% of the nucleotides in the reference sequence may be deleted or substituted with another nucleotide, or a number of nucleotides up to 5% of the total nucleotides in the refemce sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Methods to determine identity and similarity are codified in publicly available programs. Preferred computer program methods to determine identity and similarity betweeen two sequences include, but are not limited to, the GCG program package (Devereux, J., et al., Nucleic Acid Ressearch 12 (1):387 (1984)), BLASTP, BLASTN, and FASTA (Altschul, S. F., et al., J. Molec.Biol.215:403-410(1990)). The BLASTX program is publicly available from NCBI and other sources (BLAST Manual, Altschul, S. F., et al., NCBI NLM NIH Bethesda, Md. 20894; Altschul, S. F., et al., J. Molec.Biol.215:403-410(1990)).

Isolated nucleic acid molecules, as defined above, can be cDNA, genomic DNA, synthetic DNA, or RNA, and can be double-stranded or single-stranded (i.e., either a sense or an antisense strand). Fragments of these molecules, which are also considered within the scope of the invention, can be produced, for example, by the polymerase chain reaction (PCR) or generated by treatment with one or more restriction endonucleases. A ribonucleic acid (RNA) molecule can be produced by in vitro transcription.

In the present invention, Parkin cDNA was PCR amplified using the primers: 5′-GAGCTAGCCACCATGATAGTGTTTGTCAGG-3′ and 5′-GTGMTTCCTACACGTCGMCCAG-3′. The amplified Parkin cDNA was cloned into a Nhe I and EcoR I site of pcDNA3.1/Zeo(−) vector and the parkin cDNA sequence was checked by sequencing. To prepare the D126E mutant, a site-directed mutagenesis was performed by using the Quickchange Mutagenesis Kit (Stratagene) according to the manufacturers instructions. 5′-TCATTCTGCACACTGAAAGCAGGAAGGACTCACC-3′ were used for preparing the D126E mutated construct. This Vector was then transfected into SH-SY5Y or CHO cells using FUGENE6 (Boehringer Mannheim) and stable transfected cell lines were selected using 0.25 microliter Zeocin/ml medium (SH-SY5Y cells) or 3 microliter Zeocin/ml medium (CHO cells).

The nucleic acid molecules of the invention can contain naturally occurring sequences, or sequences that differ from those that occur naturally, but, due to the degeneracy of the genetic code, encode the same polypeptide. In addition, these nucleic acid molecules are not limited to sequences that only encode functional polypeptides, and thus, can include some or all of the non-coding sequences that lie upstream or downstream from a coding sequence.

The nucleic acid molecules of the invention can be synthesised (for example, by phosphoramidite-based synthesis) or obtained from a biological cell, such as the cell of a mammal. Thus, the nucleic acids can be those of a human, mouse, rat, guinea pig, cow, sheep, horse, pig, rabbit, monkey, dog, or cat. Combinations or modifications of the nucleotides within these types of nucleic acids are also encompassed.

The isolated nucleic acid molecules of the invention encompass fragments that are not found as such in the natural state. Thus, the invention encompasses recombinant molecules, such as those in which a nucleic acid sequence is incorporated into a vector (for example, a plasmid or viral vector) or into the genome of a heterologous cell (or the genome of a homologous cell, at a position other than the natural chromosomal location). These circumstances are discussed further below.

In the event the nucleic acid molecules of the invention encode or act as antisense molecules, they can e.g. be used to regulate transcription.

In most cases, a DNA sequence encoding a Parkin D126 will be one which produces Parkin D126 mRNA directly, without splicing. Thus, the DNA sequence will be based on the Parkin D126 mRNA sequence rather than the Parkin genomic sequence.

In addition to the nucleotide sequences disclosed herein (see, for example SEQ ID NO: 3), equivalent forms may be designed for other species, and can be identified and isolated by using the nucleotide sequences disclosed herein and standard molecular biological techniques. For example, homologues of Parkin may be isolated from other organisms by performing PCR, using two degenerate oligonucleotide primer pools designed on the basis of amino acid sequences of the alternatively spliced exons. Whereupon a site-directed mutagenesis is performed to generate the D126E mutant. The template for the reaction may be cDNA obtained by reverse transcription of mRNA, prepared from, for example, human or non-human cell lines or tissues, particularly those known or suspected to express Parkin. The PCR product may be subcloned and sequenced to ensure that the amplified nucleic acid sequence represents the sequence of Parkin D126.

The invention also encompasses nucleotide sequences that encode fragments of Parkin D126, and that retain at least said amino acid sequence of Parkin D126 that harbours the mutated Asp126 site, as described herein.

The present invention further relates to means of altering the proteolytical procession of wild type Parkin by a potential cleaving enzyrne at said Asp 126 or in close vicinity thereof, thereby preventing and/or reducing proteolytic processing of Parkin by a protease. In a specifically preferred embodiment, said protease is a cysteine proteinase that cleaves a substrate after an aspartic acid residue. In the present context, such proteases preferrably belong to the family of caspases. Table 1 summarises examples of members of the constantly growing family of caspases, and is in not meant to be exclusive.

TABLE 1

| | |
|---|---|
| Group I: mediators of | caspase-1 (ICE) |
| inflammation | caspase-4 ICErel-II TX, ICH-2 |
| | caspase-5 (ICErel-III, TY) |
| Group II: effectors of apoptosis | caspase-2 (ICH-1, mNEDD2) |
| | caspase-3 (apopain, CPP-32, YAMA) |
| | caspase-7 (Mch-3, ICE-LAP3, CMH-1) |
| Group III: activators of | caspase-6 (Mch2) |
| apoptosis | caspase-8 (MACH, FLICE, Mch5) |
| | caspase-9 (ICE-LAP6, Mch6) |
| | caspase-10 |

A nucleic acid sequence or a fragment of it, as encompassed in the present invention, comprising the amino acid position 126, can be used for preventing or treating PD in a patient in a multitude of ways, or for diagnosing a potential disposition of a subject for the disease, but can as well be used for treating, preventing or diagnosing other neurological disorders that are associated with neuronal loss due to caspase-associated cell death and/or apoptosis, comprising e.g. Alzheimer's disease or Huntington's disease.

The invention also encompasses: (a) expression vectors that contain any of the foregoing Parkin D126 coding sequences and/or their complements (that is, “antisense” sequence); (b) expression vectors that contain Parkin D126 coding sequences operatively associated with a regulatory element that directs the expression of the coding sequences; (c) expression vectors containing Parkin D126 nucleic acid molecules and heterologous nucleic acid molecules, such as molecules encoding a reporter or marker; and (d) genetically engineered host cells that contain any of the foregoing expression vectors and thereby express the nucleic acid molecules of the invention in the host cell.

As used herein, regulatory elements include but are not limited to inducible and non-inducible promoters, enhancers, operators and other elements, which are known to those skilled in the art, that drive and regulate gene expression. Such regulatory elements include but are not limited to the cytomegalovirus hCMV immediate early gene, the early or late promoters of SV40 adenovirus, the Jac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast.alpha.-mating factors.

Similarly, the nucleic acid of the present invention can form part of a hybrid gene encoding additional polypeptide sequences (for example, sequences that function as a marker or reporter) that can be used, for example, to produce a fusion protein (as described further below). Examples of marker or reporter genes include β-lactamase, chloramphenicol acetyltransferase (CAT), adenosine deaminase (ADA), aminoglycoside phosphotransferase (neor, G418r), dihydrofolate reductase (DHFR), hygromycin-B-phosphotransferase (HPH), thymidine kinase (TK), lacZ (encoding 13-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

The expression systems that may be used for purposes of the invention include but are not limited to microorganisms such as bacteria (for example, E. coli and B. subtilis) transformed with recombinant bacteriophage DNA, plasmid DNA or cosmid DNA expression vectors containing the nucleic acid molecules of the invention; yeast (for example, Saccharomyces and Pichia) transformed with recombinant yeast expression vectors containing the nucleic acid molecules of the invention (preferably containing the nucleic acid sequence of SEQ ID NO: 3); insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, T1 plasmid) containing Parkin D126 nucleotide sequences; or mammalian cell systems (for example, COS, CHO, BHK, 293, VERO, HeLa, MDCK, WI38, and NIH 3T3 cells) barbouring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors may be advantageously selected, depending upon the use intended for the gene product being expressed. For example, when a large quantity of such a protein is to be produced, for the generation of pharmaceutical compositions of Parkin D126, polypeptides for raising immunogenic substances, such as antibodies to those proteins, vectors that are capable of directing the expression of high levels of fusion protein products that are readily purified may be desirable. Such vectors include, but are not limited to, the E. coli expression vector pUR278 (Ruther et al., EMBO J. 2:1791, 1983), in which the coding sequence of the insert may be ligated individually into the vector in frame with the lacZ coding region so that a fusion protein is produced; piN vectors (Inouye and Inouye, Nucleic Acids Res. 13:3101-3109, 1985; Van Heeke and Schuster, J. Biol. Chem. 264:5503-5509, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to Include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The coding sequence Parkin D126 may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacing the proteinaceous coat coded for by the polyhedrin gene). These recombinant viruses are then used to infect Spodoptera frugiperda cells in which the inserted gene is expressed. (for example, see Smith et al. J. Virol. 46:584,1983; Smith, U.S. Pat. No. 4,215,051).

In mammalian host cells, a number of viral-based expression systems may be utilised. In cases where an adenovirus is used as an expression vector, the nucleic acid molecule of the invention may be ligated to an adenovirus transcription/translation control complex, for example, the late promoter and tripartite leader sequence. This chimeric gene may then be inserted in the adenovirus genome by in vitro or in vivo recombination. Insertion in a non-essential region of the viral genome (for example, region E1 or E3) will result in a recombinant virus that is viable and capable of expressing the polypeptide encoded by the nucleic acid molecule of the invention in infected hosts (for example, see Logan and Shenk, Proc. Natl. Acad. Sci. USA 81:3655-3659, 1984). Specific initiation signals may also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544, 1987).

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express Parkin D126 sequences as described above, may be engineered. Rather than using expression vectors which contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which produce Parkin D126. Such engineered cell lines (as demonstrated in FIG. 5) may be particularly useful in screening and evaluation of compounds that affect the endogenous activity of the gene product.

A number of selection systems may be used, including but not limited to the herpes simplex virus thymidine kinase (Wigler, et al., Cell 11:223,1977), hypoxanthine-guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk-, hgprt- or aprt-cells, respectively. Also, antimetabolite resistance can be used as the basis of selection for the following genes: dhfr, which confers resistance to methotrexate (Wigler, et al., Proc. Natl. Acad. Sci. USA 77:3567, 1980; O'Hare, et al., Proc. Natl. Acad. Sci. USA 78:1527,1981); gpt, which confers resistance to mycophenolic acid (Mulligan and Berg, Proc. Natl. Acad. Sci. USA 78:2072,1981); neo, which confers resistance to the aminoglycoside G-418 (Colberre-Garapin et al., J. Mol. Biol. 150:1,1981); and hygro, which confers resistance to hygromycin (Santerre et al., Gene 30: 147, 1984).

Alternatively, any fusion protein may be readily purified by utilising an antibody specific for the fusion protein being expressed. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni2+.cndot.nitriloacetic acid-agarose columns and histidine-tagged proteins are selectively eluted with imidazole-containing buffers.

Polypeptides of the Invention

The present invention further encompasses a purified polypeptide having an amino acid sequence that is at least 70% identical to an amino acid sequence such as shown in SEQ ID NO: 4, or a fragment of said amino acid sequence that is at least 70% identical to a fragment of an amino acid sequence such as shown in SEQ ID NO: 4, including a point mutation at Asp 126, wherein at least it differs from the wild type amino acid sequence such as shown in SEQ ID NO: 2, and which is functionally homologous to a mutated Parkin polypeptide or a fragment of said polypeptide, wherein said point mutation at Asp 126 alters a potential cleavage site of said polypeptide. Said point mutation at Asp 126 prevents proteolytic processing of said polypeptide in a mammalian cell by a protease, and in an especially preferred embodiment, said protease is a cysteine proteinase that cleaves a substrate after an aspartic acid residue comprising a protease belonging to the family of caspases as listed in Table 1.

In the present context, a purified polypeptide having an amino acid sequence that is at least 70% identical comprises sequence identities such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or at least 100% identical to an amino acid sequence such as shown in SEQ ID NO: 4, or a fragment of said amino acid sequence, respectively.

By a polypeptide having an amino acid sequence at least, for example, 95% identical to a reference amino acid sequence as shown in SEQ ID No.4, is intended that the amino acid sequence of the polypeptide is identical to the reference sequence except that the amino acid sequence may include up to 5 point mutations per each 100 amino acids of the reference amino acid sequence as shown in SEQ ID No.4. In other words, to obtain a polypeptide having an amino acid sequence at least 95% identical to a reference amino acid sequence: up to 5% of the amino acids in the reference sequence may be deleted or substituted with another amino acid, or a number of amino acids up to 5% of the total amino acids in the refemce sequence may be inserted into the reference sequence. These mutations of the reference sequence may occur at the amino or carboxy terminal positions of the reference amino acid sequence or anywhere between those terminal positions, interspersed either individually among amino acids in the reference sequence or in one or more contiguous groups within the reference sequence.

Methods to determine identity and similarity are codified in publicly available programs. Preferred computer program methods to determine identity and similarity betweeen two sequences include, but are not limited to, the GCG program package (Devereux, J., et al., Nucleic Acid Ressearch 12 (1):387 (1984)), BLASTP, BLASTN, and FASTA (Altschul, S. F., et al., J. Molec.Biol 0.215:403-410(1990)). The BLASTX program is publicly available from NCBI and other sources (BLAST Manual, Altschul, S. F., et al., NCBI NLM NIH Bethesda, Md. 20894; Altschul, S. F., et al., J. Molec.Biol.215:403-410(1990)).

The Parkin D126 polypeptides described herein and fragments, mutants, and truncated forms thereof, including fusion proteins, can be prepared for a variety of uses, including but not limited to the generation of antibodies, as reagents in diagnostic assays, for the identification of other cellular gene products involved in the regulation of apoptosis, as reagents in assays for screening for compounds that can be used in the treatment of disorders associated with apoptotic cell death, abnormal activity of Parkin, or abnormal activity of caspases, and as pharmaceutical reagents useful in the treatment of neurological disorders that are associated with neuronal cell loss associated with caspases and/or apoptosis.

The present invention takes in proteins and polypeptides that may have one or more of the functions of naturally-occurring Parkin but which at least differ from wild type Parkin in having an altered or deleted potential cleavage site at amino acid position number 126, which alters or obstructs proteolytical procession of Parkin at said potential cleavage site and/or in the close proximity thereof. Among the functional attributes of wild type Parkin are the protection of a cell from toxic stress comprising unfolded protein stress and/or endoplasmatic reticulum (ER) stress and inhibition of a certain type of cell death through proteasome mediated protein degradation.

Polypeptides having one or more functions of naturally-occuring Parkin can be closely related to Parkin D126. Such polypeptides can be created by functionally equivalent proteins and include, but are not limited to, additions or substitutions of amino acid residues within the amino acid sequence encoded by the nucleotide sequence described above (see SEQ ID NO: 3), but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved.

While random mutations can be made to Parkin D126 DNA (using random mutagenesis techniques well known to those skilled in the art) and the resulting mutant polypeptides tested for activity, site-directed mutations of these coding sequence can be engineered (using site-directed mutagenesis techniques well known to those skilled in the art) to generate mutant polypeptides with increased function, for example greater ability to inhibit apoptotic cell death.

While the polypeptides of the invention can be chemically synthesised (for example, see Creighton, “Proteins: Structures and Molecular Principles,” W. H. Freeman & Co., N.Y., 1983), large polypeptides, may advantageously be produced by recombinant DNA technology including in vitro recombinant DNA techniques, synthetic techniques, and in vivo genetic recombination described herein. In addition, skilled artisans may consult Ausubel et al. (“Current Protocols in Molecular Biology, Vol. I,” Green Publishing Associates, Inc., and John Wiley & sons, Inc., N.Y., 1989), Sambrook et al. (“Molecular Cloning, A Laboratory Manual,” Cold Spring Harbor Press, Cold Spring Harbor, N.Y., 1989), and, particularly for examples of chemical synthesis, Gait, M. J. (Ed. “Oligonucleotide Synthesis,” IRL Press, Oxford, 1984), which are incorporated by reference herein in their entirety.

Antibodies

The invention also relates to an immunogenic substance that reacts with a polypeptide according to any of claims 10-16 and/or claim 22.

The invention further relates to an immunogenic substance that selectively binds to a polypeptide fragment of Parkin, wherein the said fragment is the result of cleavage of Parkin at Asp 126. Preferably, such immunogenic substances selectively bind to a region of said cleaved Parkin polypeptide fragment, located within 50, preferably 40, more preferably 30, more preferably 20, most preferably 10 amino acids from Asp126 on either side thereof. It is believed that the said regions comprise epitopes, to which antibodies specific to the polypeptide fragments formed by the cleavage of Parkin at Asp 126 bind.

In particular, the invention relates to an immunogenic substance that reacts with a peptide corresponding to SEQ ID NO: 5 and/or SEQ ID NO: 6. It is believed that the sequences of SEQ ID NO: 5 and/or SEQ ID NO: 6 constitute epitope regions or a part thereof, to which antibodies specific to the polypeptide fragments formed by the cleavage of Parkin at Asp 126 bind.

Also, the invention relates to an immunogenic substance, wherein the polypeptide fragment resulting from the cleavage of Parkin at Asp 126 has a changed conformational structure compared to the original conformational structure of the corresponding uncleaved Parkin, and wherein said immunogenic substance binds to said polypeptide fragment having a changed conformational structure. Thus, it is believed that said changed conformational structure facilitates the selective binding of said immunogenic substance to said polypeptide fragment of Parkin. As is well known in the art, a cleavage of any protein will usually result in a change of the conformational structure of the polypeptide fragment compared to the same region in the original protein. The physical separation of polypeptide fragments of Parkin at Asp126 will thus generate a new and changed conformational structure in said polypeptide fragments, due to e.g, but not limited to, breaks of disulphide bonds and van-der-Waals forces.

The invention also encompasses immunogenic substances, such as antibodies that bind Parkin that has been cleaved after the potential cleavage site at Asp 126 or in the close vicinity thereof. Antibodies that specifically recognise one or more epitopes of this protein, or fragments thereof are also encompassed by the invention. Such antibodies include but are not limited to polyclonal antibodies, monoclonal antibodies (mAbs), humanised or chimeric antibodies, single chain antibodies, Fab fragments, F(ab′)₂ fragments, fragments produced by a Fab expression library, anti-idiotypic (anti-Id) antibodies, and epitope-binding fragments of any of the above. This antibody will be especially useful for a variety of purposes, including for detecting Parkin cleavage, monitoring disease progression and screening of substances for their ability to inhibit Parkin cleavage in vitro and in vivo.

The antibodies of the invention may be used, for example, in the detection of various forms of cleaved Parkin that has been cleaved after the potential cleavage site at Asp 126 or in the close vicinity thereof, as described above, in a biological sample and may, therefore, be utilised as part of a diagnostic or prognostic technique whereby patients may be tested for abnormal amounts of Parkin that is processed at amino acid position 126 or in close vicinity thereof. Such antibodies may also be utilised in conjunction with, for example, compound screening schemes, as described below, for the evaluation of the effect of test compounds on Parkin and/or Parkin D126. Additionally, such antibodies can be used in conjunction with the gene therapy techniques described below, to, for example, evaluate cells expressing the alternate forms described herein prior to their introduction into the patient.

For the production of antibodies, various host animals may be immunised by injection with a peptide having a sequence that is present in Parkin that has been cleaved after the potential cleavage site at Asp 126 or in the close vicinity thereof. Such host animals may include but are not limited to rabbits, mice, and rats, to name but a few. Various adjuvants may be used to increase the immunological response, depending on the host species, including but not limited to Freud's (complete and incomplete), mineral gels such as aluminium hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunised animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497,1975; and U.S. Pat. No. 4,376,110), the human B cell hybridoma technique (Kosbor et al., Immunology Today 4:72, 1983; Cole et al., Proc. Natl. Acad. Sci. USA 80:2026-2030, 1983), and the EBV-hybridoma technique (Cole et al., “Monoclonal Antibodies And Cancer Therapy,” Alan R. Liss, Inc., pp. 77-96,1985). Such antibodies may be of any immunoglobuline class including IgG, IgM, IgE, IgA, IgD and any subclass thereof. The hybridoma producing the mAb of this invention may be cultivated in vitro or in vivo. Production of high titres of mAbs in vivo makes this the presently preferred method of production.

In addition, techniques developed for the production of “chimeric antibodies” (Morrison et al., Proc. Natl. Acad. Sci. USA, 81:6851-6855, 1984; Neuberger et al., Nature, 312:604-608, 1984; Takeda et al., Nature, 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobuline constant region.

Alternatively, techniques described for the production of single chain antibodies (U.S. Pat. No. 4,946,778; Bird, Science 242:423-426,1988; Huston et al., Proc. Natl. Acad. Sci. USA 85:5879-5883, 1988; and Ward et al., 1989, Nature 334:544-546,1989) can be adapted to produce single chain antibodies against caspase-8h or caspase-8i gene products. Single chain antibodies are formed by linking the heavy and light chain fragments of the Fv region via an amino acid bridge, resulting in a single chain polypeptide.

Furthermore, antibody fragments which recognise specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., Science, 246:1275-1281, 1989) to allow rapid and easy identification of monoclonal Fab fragments with the desired specificity.

Antibodies can be humanised by methods known to those skilled in the art. For example, monoclonal antibodies with a desired binding specificity can be commercially humanised (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

The methods described herein may be performed, for example, by utilising pre-packaged diagnostic kits comprising at least one specific Parkin nucleotide or peptide sequence or antibody reagent described herein, which may be conveniently used, for example, in clinical settings, to diagnose patients exhibiting symptoms of the disorders described below.

Inhibiting and/or Altering Proteolytical Processing of Parkin in a Neuronal Cell

A plausible scenario in the pathology of PD is that environmental factors, genetic disposition or other physiological factors provoke activation of cellular stress responses, comprising the partial activation of the endogenous caspase-dependent cell suicide pathway in susceptible neurones. The cells attempt to restore the cellular homeostasis by activating cellular defense programs as exemplified by the unfolded protein stress response wherein Parkin plays a cytoprotective role. Activated caspases inhibit Parkin function by proteolytic cleavage and thus lower the cytoprotective potential of the cells. The hereby sensitised cells, in turn, produce elevated levels of caspases such as for example caspase 1 that acts slowly as a chronic initiator and caspase-3 acting as the final effector of cell-death, leading to exacerbation of a vicious cycle and the sequential activation of additional caspases that part-take in the toxic cascade, which culminates in neuronal loss. Thus, components of the apoptotic machinery contribute either directly or indirectly to the complex proteolytic processing of Parkin that in its turn ultimately leads to the death of neurones and the development of PD.

Caspase-3 is an effector of apoptosis in experimental models for PD. A positive correlation has been shown between the degree of neuronal loss in dopaminergic (DA) cell groups affected in the mesencephalon of PD patients and the percentage of caspase-3 positive cells. This suggests that neurones expressing caspase-3 are more sensitive to the pathological process of PD. It could further be shown that PD SN neurones express more caspase-3 than SN neurones from healthy subjects and that caspase-3 activation precedes and is not a consequence of apoptotic cell death in PD (Hartmann et al., PNAS, March 14 2000, vol 97, no.6, 2875-2880). In light of this, the findings of the present invention clearly indicate that a caspase, most probably caspase-3, cleaves Parkin preceding apoptotic cell death. Thus, the possibility to prevent cleavage of Parkin by caspase-3 or any other caspase taking part in the toxic cascade will prevent PD, or at least have beneficial effect on the outcome of the disease.

As the cleavage of Parkin abrogates the cytoprotective function of Parkin in the cell, the inhibition of Parkin cleavage at Asp 126 or in the close vicinity thereof, represents a novel and exiting possibility to alter the successive loss of Parkin in PD and thereby halting or delaying the successive accumulation of toxic stress on the cell, which will otherwise ultimately result in the loss of neurones in PD patients. The present invention therefor relates to different methods for inhibiting and/or altering Parkin cleavage and/or proteolytic processing at said specific potential cleavage site at Asp126.

Cell surface receptors (e.g., CD95/APO-1/Fas; TNF receptor) and their ligands (CD95-L; TNF) as well as evolutionarily conserved mechanisms involving proteases, mitochondrial factors (e.g., Bcl-2-related proteins, reactive oxygen species, mitochondrial membrane potential, opening of the permeability transition pore), endoplasmic reticulum associated initiators of the unfolded protein stress response, or p53 participate in the modulation and execution of cell death. Triggers comprise oxidative stress, inflammatory processes, calcium toxicity and survival factor deficiency. Therapeutic agents are being developed to interfere with these events, thus conferring the potential to be neuroprotective. In this context, drugs with anti-oxidative properties, e.g., flupirtine, N-acetylcysteine, idebenone, melatonin, but also novel dopamine agonists (ropinirole and pramipexole) have been shown to protect neuronal cells from apoptosis and thus have been suggested for treating neurodegenerative disorders like AD or PD. Other agents, like non-steroidal anti-inflammatory drugs (NSAIDs) partly inhibit cyclooxygenase (COX) expression, as well as having a positive influence on the clinical presentation of AD. Distinct cytokines, growth factors and related drug candidates, e.g., nerve growth factor (NGF), or members of the transforming growth factor-beta (TGF-beta) superfamily, like growth and differentiation factor 5 (GDF-5), protect tyrosine hydroxylase or dopaminergic neurones from apoptosis. Furthermore, peptidergic cerebrolysin has been found to support the survival of neurones in vitro and in viva. Treatment with protease inhibitors is suggested as potential targets to prevent DNA fragmentation in dopaminergic neurones of PD patients. Finally, CRIB (cellular replacement by immunoisolatory biocapsule) is an auspicious gene therapeutical approach for human NGF secretion, which has been shown to protect cholinergic neurones from cell death when implanted in the brain.

In one embodiment of the present invention, a small organic compound is used that specifically binds to a potential cleavage site of Parkin at Asp 126 in an amino acid sequence such as shown in SEQ ID NO: 2, or in close proximity thereof and thus alters the accessibility of said site to a potential cleaving enzyme, for manufacturing a pharmaceutical composition for treating and/or preventing a neurological disorder in a mammal. Said small organic compound can alternatively acts as a ligand for a protease and/or as an activator of a protease inhibitor, thus altering the proteolytic processing of Parkin at the potential cleavage site of said Parkin at Asp 126. Relating to this specific aspect of the present invention, “close proximity” means that a small organic compound binds to a potential cleavage site of Parkin at Asp 126 in an amino acid sequence such as shown in SEQ ID NO: 2, or to any other amino acid residue which is at most 1-50 amino acid residues N-terminal or C-terminal from said site, such as at most 1, 2, 3, 4, 5, 6, 7,8,9,10, 11, 12, 13, 14, 15, 16, 17, 18, 19, 20, 21, 22,23,24, 25, 26, 27, 28, 29, 30, 31, 32, 33, 34, 35, 36, 37, 38, 39, 40, 41, 42, 43, 44, 45, 46, 47, 48, 49, or 50 amino acids away in either direction.

In the present context, such substances and/or organic compounds are comprised in the group consisting of peptide and non-peptide inhibitors of caspases and caspase activators and peptide and non-peptide ligands for Parkin that prevent the cleavage of Parkin after Asp 126. Small organic compounds (SOC) and peptides comprised by the current invention are organic molecules that modulate the cleavage of parkin after D1 26.

The SOC and peptides may bind directly to Parkin, thereby changing its properties as a protease substrate. Alternatively, the SOC and peptides may bind to enzymes that act on Parkin. The action may fascilitate or inhibit the action of the enzymes thereby i) modulating the activity of protease's reactivity toward Parkin or ii) modulating the activity of kinases and phosphatases that act on Parkin or iii) modulating other enzymes that may modify Parkin, such as e.g. ubiquitin ligases.

SOCs are small compounds and peptides ranging in size from MW units of 100 to 5000. SOCs are often made by combinatorial chemistry and their chemical natures are as such legio. Examples of groups of compounds used for the synthesis of SOCs by combinatorial chemistry are arylhydrazines, aminoheterocycles, 4-arylthiazoles, 2-formylthiazoles, phenylacetic acids, tryptamines, 2-indolecarboxylic acids, 3-indolylacetic acids, diaryl ethers, isonitriles, pyrrolcarbaldehydes, 2-substituted pyrrolidines, 2-substituted piperidines, 2-substituted azepanes and N-arylpyrroles.

The interaction between the SOC/peptides and their above described targets can be reversible or irreversible. Examples of such compounds can in principle apply to inhibitors of many groups of enzymes. Examples are listed below for members from the groups of

1) protease inhibitors; organophosphates, sulphonyl fluorides, coumarins and related heterocyclic compounds and peptides derivatised as aldehydes, boronic acids, chloromethyl ketones, diazomethanes and epoxides

2) kinase inhibitors; phenothiazines, naphthalene sulfonamides, tyrphostin, staurosporine and calphostin C

3) phosphatase inhibitors; okadaic acid and microcystin.

SOCs and peptides used for screening for their activities towards Parkin cleavage after D126 can be identified by the in vivo screening of cellular assays or in vitro screening for Parkin cleavage in isolated systems. As is well known by the skilled artisan, SOCs and peptides for the purposes comprised in the present invention are often present in small-molecule libraries and peptide libraries. The latter are commercially available. The formers are often the property of companies but smaller libraries are also commercially available e.g. from ACD chemicals (http://www.acdchemicals.com/).

In another embodiment of the present invention, a small peptide or peptide fragment is used, which acts as a protease inhibitor, as a ligand for a protease and/or as an activator of a protease inhibitor, thus altering the proteolytic processing of Parkin at the potential cleavage site of said Parkin at Asp 126 in an amino acid sequence such as shown in SEQ ID NO: 2, for manufacturing a pharmaceutical composition for treating and/or preventing a neurological disorder in a mammal. In the context of the present invention, a small peptide comprises 20 amino acids or less, such as 3 amino acids, 4, 5, 6, 7, 8, 9, 10, 11, 12, 13, 14, 15, 16, 17, 18, 19, or 20 amino acids. A small peptide included in the present invention is e.g. a tetrapeptide based on the Parkin sequence N-terminal to the cleavage site, which reads Leu-His-Thr-Asp.

Still another aspect of the present invention relates to the use of inhibitors and/or activators of a kinase and/or a phosphatase for altering the proteolytic processing of Parkin at the potential cleavage site of said Parkin at Asp 126 in an amino acid sequence such as shown in SEQ ID NO: 2, for manufacturing a pharmaceutical composition for treating and/or preventing a neurological disorder in a mammal.

Kinases and phosphatases are commonly grouped according to their specificity in phosphorylation/dephosphorylation of Ser/Thr and Tyr residues, respectively. Below members of the different groups are listed as examples of enzymes that can be used for the regulation of Parkin cleavage. The table is not exhaustive but does represent examples of enzymes that can be employed in methods as described in the present invention.

TABLE 2

| | | |
|---|---|---|
| Kinases: | Ser/Thr | Stress activated protein kinase/c-jun N-terminal |
| | | kinase pathway |
| | | Extracellular signal-regulated kinase/mitogen |
| | | activated protein (MAP) kinase pathway, |
| | | G-protein-coupled receptor kinases, protein |
| | | kinase A, Protein kinase C family, calmodulin- |
| | | dependent kinases, cyclin-dependent kinases |
| | Tyr | Growth factor receptor-associated kinases, e.g. |
| | | PDGF receptor, insulin receptor, Src-family of |
| | | protein tyrosin kinases, e.g. c-src, Abl, Fyn, |
| | | Janus |
| Phosphatases: | Ser/Thr | protein phosphatase 2A, protein phosphatase |
| | | 2C, MAP kinase phosphatases (dual specificity) |
| | Tyr | MAP kinase phosphatases, hemapoietic |
| | | phosphatases e.g. STEP, PCPTP1 |

Methods for Screening of Organic Compounds for Their Ability to Inhibit Parkin D126 Cleavage in Cell Free and Cell Based Assays.

Inhibition of Parkin cleavage after amino acid position126 represents a therapeutic target that will offer neuroprotection in Parkinson's disease and other neurodegenerative diseases. The screening of such inhibitors can be pursued in cell-free and cell-based assays.

A cell free assay can be performed as follows. First a substrate has to be synthesised based on the Parkin sequence around the D126 cleavage site. The substrate could be a synthetic peptide, such as for example SVGLAVILHTDSRKDSPP, corresponding to Parkin 117-133, with an attachment molecule, e.g. biotin, attached to one end and a reporter molecule e.g. a fluorochrome attached to the other end. The substrate is immobilised on streptavidin coated microtiter plates and incubated with e.g., cell extracts or purified proteases. The cell extracts can then be treated with substances that activate Parkin-cleaving proteases, e.g. staurosporine or okadaic acid in the absence or presence of the organic compounds to be tested. Compounds that inhibit and/or alter the cleavage will increase the fluorescense signal from the plates as compared to the negative control, wherein cleavage occurs. Control experiments can be performed, wherein the D126 in the peptide has been changed, to verify that the cleavage indeed takes place after D126. Other methods based on the principle of monitoring in vitro Parkin cleavage after D126 are also embodied by this description, e.g. with different lengths of Parkin peptides, different chemistries for immobilising and monitoring cleavage, different cellular extracts, purified components, or by e.g. using the antibody that recognises Parkin cleaved after D126 (as described in example 3 and 6).

A cell based assay can be performed as follows. Cell lines expressing human Parkin, as described in example 5, are cultured on microtiter plates and challenged to substances that induce cleavage of Parkin after D126, e.g. staurosporine, okadaic acid, tunicamycin or mercaptoethanol. Some of the cells are concomitantly treated with the organic compounds to be tested. The cells will be fixed in e.g. 4% paraformaldehyde after incubation for a time that permits Parkin cleavage in untreated cells, and will be processed for immunofluorescense microscopy using an antibody that specifically recognises Parkin which is cleaved after D126 (described in example 3 and 6). As negative controls cells are used wherein amino acid position 126 has been changed in order to inhibit the specific cleavage after this residue. Other methods, based on the principle of monitoring in vivo Parkin cleavage after D126 are also embodied by this description, e.g. by comparing the levels of cleavage using immunoblotting techniques, thereby circumventing the need for a Parkin cleavage specific antibody.

Characterising and Monitoring the Susceptibility of a Patient's Parkin to Cleavage After D126 by the Study of Primary Cell Cultures.

Neurodegenerative disorders like Parkinson's disease are likely to be heterogeneous in terms of them inducing events and the cellular factors that sustain the degenerative phenotype, e.g. a generalised complex I deficiency in the mitochondria of some Parkinson's disease patients. It will be beneficial to evaluate the patients own cellular Parkin expression in their own cellular milieu and driven by its own promoter as this will enable the testing of Parkin in relation to patient-specific cellular stress and/or to the monitoring of treatment responses. Unfortunately, Parkin expression in non-central nervous tissue is very low or absent. To circumvent this problem cultures of accessible patient cells, e.g. dermal fibroblast from skin biopsies and isolated peripheral leukocytes will be cultured in the presence of low levels of mercaptoethanol to induce a sublethal unfolded protein stress. This will induce the expression of Parkin, as part of the unfolded protein response, and enable its study in cells and cell extracts by biochemical and immunochemical means. For example, Parkin stability, turn-over and posttranslational modifications can thus be monitored after immunoblotting and/or metabolic labeling.

Assays

An assay for apoptosis wherein proteolytic cleavage of Parkin takes place can be used in screening assays to identify compounds that increase or decrease apoptosis. The compounds identified by using these assays may alter in the level of apoptosis, by modulating the proteolytic cleavage of Parkin. Compounds identified in these assays can be used as therapeutic compounds to treat neurological disorders.

Assays for detecting apoptosis wherein proteolytic cleavage of Parkin takes place generally employ Parkin-expressing cells. The cells can be those of stably transfected cell lines or cell cultures that normally express Parkin.

Numerous substances are capable of inducing apoptosis in various cell types and can thus be used in assays of apoptosis. However, Parkin protects specifically against unfolded protein stress. Therefore, substances eliciting this kind of stress will preferably be employed, such as e.g. reducing agents like mercaptoethanol or inhibitors of normal glycosylation like tunicamycin. Other apoptosis inducing substances that may be used include physiological activators, such as TNF family members, TGF-β, the neurotransmitters glutamate, dopamine, and NMDA (N-methyl-D-aspartate), calcium, and glucocorticoids. Cell death can also be induced when growth factors are withdrawn from the medium in which the cells are cultured. Additional inducers of apoptosis include heat shock, viral infection, bacterial toxins, expression of the oncogenes myc, rel, and E1 A, expression of tumour suppresser genes, cytolytic T cells, oxidants, free radicals, gamma and ultraviolet irradiation, B-amyloid peptide, ethanol, and chemotherapeutic agents such as Cisplatin, doxorubicin, arabinoside, nitrogen mustard, methotrexate, and vincristine.

Use of the Nucleic Acids, Polypeptides, and Antibodies of the Invention in the Diagnosis and Treatment of Neurological Disorders Associated with Caspase-associated Neurodegeneration and/or Apoptotic Cell Death

As described below, the nucleic acids, polypeptides, antibodies, and other reagents of the invention can be used in the diagnosis and treatment of disorders associated with caspase-associated nervecell degeneration and apoptotic cell death.

The analysis of cells taken from culture may be a necessary step in the assessment of cells to be used as part of a cell-based gene therapy technique or, alternatively, to test the effect of compounds on the expression of Parkin D126 and/or on the proteolytic processing of either Parkin D126 or wild type Parkin. Such analyses may reveal both quantitative and qualitative aspects of the expression pattern of these forms of Parkin, including activation or inactivation of their expression and the proteolytic processing of Parkin after D126.

Where a sufficient quantity of the appropriate cells can be obtained, standard Northern blot or RNAse protection analyses can be performed to determine the level of mRNA encoding the various forms of Parkin.

It is also possible to base diagnostic assays and screening assays for therapeutic compounds on detection of the Parkin polypeptides cleaved after D126. Such assays for Parkin polypeptides cleaved after D126, or peptide fragments thereof will typically involve incubating a sample, such as a biological fluid, a tissue extract, freshly harvested cells, or lysates of cells which have been incubated in cell culture, in the presence of a detectably labelled antibody capable of identifying these gene products (or peptide fragments thereof, and detecting the bound antibody by any of a number of techniques well-known in the art. Furthermore, diagnostic assays and screening assays as described above also enable the detection of differences in proteolytic processing of either Parkin D126 or wild type Parkin in said sample with regards to the potential cleavage of one or both of them at amino acid position number 126.

The biological sample may be brought in contact with and immobilised onto a solid phase support or carrier such as nitro-cellulose, or other solid support which is capable of immobilising cells, cell particles, or soluble proteins. The support may then be washed with suitable buffers followed by treatment with the detectably labelled antibody or fusion protein. The solid phase support may then be washed with the buffer a second time to remove unbound antibody or fusion protein. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of antibody against uncleaved Parkin D126, Parkin D126 cleaved at amino acid position 126 and/or wild type Parkin cleaved at amino acid position 126 for fusion proteins containing these polypeptides may be determined according to well known methods. Those skilled in the art will be able to determine operative and optimal assay conditions for each determination by employing routine experimentation.

With respect to antibodies, one of the ways in which the antibody of the instant invention can be detectably labelled is by linking it to an enzyme for use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.; Voller, A. et al., 1978, J. Clin. Pathol. 31:507-520; Butler, J. E., 1981, Meth. Enzymol. 73:482-523; Maggio, E. (ed.), “Enzyme Immunoassay,” CRC Press, Boca Raton, Ra., 1980; Ishikawa, E. et al., (eds.), “Enzyme Immunoassay,” Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect Parkin D126 through the use of a radioimmunoassay (RIA) (see, for example, Weintraub, B., “Principles of Radioimmunoassays, Seventh Training Course on Radioligand Assay echniques,” The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labelled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labelled using fluorescence emitting metals such as 152 Eu, or others of the lanthanide series. These metals can be attached to the antibody using such metal chelating groups as diethylenetriaminepentacetic acid (DTPA) or ethylenediaminetetraacetic acid (EDTA).

In a preferred embodiment, antibody can further be detectably labelled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. As demonstrated in examples 2,3 and 6, a primary antibody is incubated with a second antibody (such as horse radish peroxidase conjugated swine anti rabbit serum) for 1 hour and rinsed, where after the bound antibody is visualised by chemilumenescense.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Still further, the invention encompasses methods and compositions for the treatment of the neurological disorders described above, and any other neurological disorders that are found to be associated with caspase-associated nervecell degeneration and/or apoptotic cell death. Such methods and compositions are capable of modulating the level of expression or cleavage of Parkin D126 and/or wild type Parkin.

Numerous ways of altering the expression, post-translational procession or activity of the polypeptides of the invention are known to skilled artisans. For example, living cells can be transfected in vivo with the nucleic acid molecules of the invention (or transfected in vitro and subsequently administered to the patient). For example, cells can be transfected with plasmid vectors by standard methods including, but not limited to, liposome-polybrene-, or DEAE dextran-mediated transfection (see, e.g., Feigner et al., Proc. Natl. Acad. Sci. USA 84:7413, 1987; Ono et al., Neurosci. Lett. 117:259, 1990; Brigham et al., Am. J. Med. Sci. 298:278, 1989), electroporation (Neumann et al., EMBO J. 7:841, 1980), calcium phosphate precipitation (Graham et al., Virology 52:456, 1973; Wigler et al., Cell 14:725,1978; Feigner et al., supra) microinjecfion (Wolff et al., Science 247:1465,1990), or velocity driven microprojectiles (“biolistics”).

The nucleic acid molecules of the invention can be administered so that expression of the Parkin D126 occurs in tissues where wild type Parkin does not normally occur, or is enhanced in tissues where wild type Parkin is normally expressed. This application can be used for example, to suppress caspase-associated nervecell degeneration and/or apoptotic cell death and thereby treat neurological disorders in which cellular populations are diminished. Preferably, the therapeutic nucleic acid (or recombinant nucleic acid construct) is applied to the site where cells are at risk of dying by caspase-associated cell death and/or apoptotic cell death, such as directly into the substantia nigra, to the tissue in the larger vicinity, or to the blood vessels supplying the CNS.

Therapeutic Compositions

The invention features therapeutic compositions that contain the nucleic acid molecules, polypeptides, and antibodies of the invention, as well as compounds that are discovered, as described below, to affect them.

The nucleic acid molecules encoding Parkin D126, the polypeptides themselves, antibodies that specifically bind wild type Parkin, cleaved or uncleaved, or Parkin D126 that is uncleaved at amino acid position 126, and compounds that affect the expression, activity or proteolytical processing of Parkin at D126 or substances that alter the accessibility of the potential cleavage site of wild type Parkin, or that alter the rate of proteolytically cleaved wild type Parkin can be administered to a patient at therapeutically effective doses to treat or ameliorate neurological disorders associated with caspase-associated nervecell degeneration and/or apoptotic cell death. A therapeutically effective dose refers to the dose that is sufficient to result in amelioration of symptoms or decreased rate of progression of neurological disorders associated with apoptotic cell death.

Effective Dose

Toxicity and therapeutic efficacy of a given compound can be determined by standard pharmaceutical procedures, using either cells in culture or experimental animals to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimise potential damage to unaffected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilised. For any compound used in the method of the invention, the therapeutically effective dose can be estimated initially from cell culture assays. A dose may be formulated in animal models to achieve a circulating plasma concentration range that includes the IC50 (that is, the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations and Use

Pharmaceutical compositions suited for injection or implantation may be prepared by the skilled person according to conventional methods (see e.g. Scientia Medicinalis, 1997, Scherng AG, ISSN 1433-190X). The pharmaceutical compounds of the present invention may thus be formulated for ampoules, pre-filled syringes, small volume infusion containers, etc. The compositions may take such forms as suspensions in oily or aqueous vehicles, and may contain formulatory agents such as suspending, stabilising and/or dispersing agents.

The neurological disorders contemplated according to the present invention are e.g. ischemic stroke; metabolic disease of the brain; axonal injury; spinal cord injury; Alzheimer's disease; Lewy body variant of Alzheimer's disease; Multiple system atrophy; Amyothropic Lateral Sclerosis (ALS); Parkinson's disease; Huntington's disease; motor neurone disease; central nervous system infections; epilepsy; post polio syndrome; mucopolysaccharidoses (MPS), in particular MPS types I to VII; lipidosis, in particular Gaucher's disease; Lesch-Nyhan syndrome; X-linked ADL; metachromatic leukodystrophy; Krabbe's disease; Charcot-Marie-Tooth disease; Fragile X; epilepsy; Down's syndrome; phenylketonuria; or mental disorders.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurised packs or a nebulizer, with the use of a suitable propellant, for example, dichlorodifluoromethane, trichlorofluoromethane, dichlorotetrafluoroethane, carbon dioxide or other suitable gas. In the case of a pressurised aerosol the dosage unit may be determined by providing a valve to deliver a metered amount. Capsules and cartridges of, for example, gelatine for use in an inhaler or insufflator may be formulated containing a powder mix of the compound and a suitable powder base such as lactose or starch.

The compounds may be formulated for parenteral administration by injection, for example, by bolus injection, intravenous injection, intracranial injection, by administration to the cerebrospinal fluid or continuous infusion. Formulations for injection may be presented in unit dosage form, for example., in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, for example, sterile pyrogen-free water, before use.

The compounds may also be formulated in rectal compositions such as suppositories or retention enemas, for example, containing conventional suppository bases such as cocoa butter or other glycerides.

In addition to the formulations described previously, the compounds may also be formulated as a depot preparation. Such long acting formulations may be administered by implantation or by injection into the CNS. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or excipient, many of which are known to skilled artisans. Excipients which can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration.

It is well known in the medical arts that dosages for any one patient depend on many factors, including the general health, sex, weight, body surface area, and age of the patient, as well as the particular compound to be administered, the time and route of administration, and other drugs being administered concurrently.

Dosages for the polypeptides and antibodies of the invention will vary, but a preferred dosage for intravenous administration is approximately 0.0001 mg to 1 mg/ml blood volume.

Determination of the correct dosage within a given therapeutic regime is well within the abilities of one of ordinary skill in the art of pharmacology.

Use of Parkin(D126) Mutant for Gene Transfer Aiming at i) Increasing the Survival of Transplanted Genetically Engineered Cells or ii) Increasing the Survival of the Patients Neurones by Virally Transfecting them With a Parkin Mutant Resistant to Caspase Mediated Cleavage.

Therapeutically active polypeptides may be delivered to the central nervous system (CNS) by implantation of polypeptide producing cells, or by injection of viral vectors capable of infecting brain cells and directing the expression of a therapeutically active polypeptide encoded by the viral vector in the brain cells.

One strategy encompassed in the present invention is the transplantation of supply cells that i) replace the degenerating cells or ii) provide trophic support for the cells at risk or those already exhibiting degenerative traits. Herein, cells are transplanted (such as stem cells, terminally differentiated cell lines or trophic factor producing cells) that express Parkin D126. As Parkin is a factor that is important for the survival of neurones and that protects cell lines against unfolded protein stress, expressing a Parkin protein resistant to caspase cleavage after Asp126, renders the cell enhanced cytoprotective potential. A general problem in this context, the viability loss of transplanted cells, can thus be circumvented by transfecting the cells to be transplanted with a vector that drives the expression of a non-cleavable human Parkin mutant. Accordingly, making the transplanted cells express D126 mutated Parkin protein provides a basis for circumventing problems with viability loss.

A second strategy is to make the nerve cells at risk or those already exhibiting degenerative traits express D126 mutated Parkin. This can be done by constructing viral expression vectors that can target the vulnerable cells e.g. lentiviral vectors (Kordower J H et al., 2000, Neurodegeneration prevented by lentiviral vector delivery of GDNF in primate models of Parkinson's disease, Science 290:767-73). The viral vectors will, upon infection of the target cells, drive the expression of the Parlkin protein and thereby make them more resistant to cellular stress as demonstrated by a decreased rate of degeneration.

Neuronal Implantation

Various ways of achieving therapy of the CNS using intracerebral implantation of genetically modified cells and the use of viral vectors for the delivery of 20 therapeutic genes to the CNS has been considered.

According to one approach, primary and immortalised cells have been successfully used for gene transfer applications. Examples of such cells are primary cells of neuronal origin (e.g. glia cells and astrocytes), or of non-neuronal origin (e.g. fibroblasts, myoblasts and hepatocytes). Such cells may not survive for longer periods in the CNS, unless they are immortalised. Intracerebral grafting of foetal tissue for the treatment of neurological disorders has also been investigated. Alternatively stem cells may be used.

Another cell type which has been proposed as gene transfer vehicle is the cerebral endothelial cell. The direct implantation of immortalised and genetically so transformed cerebral endothelial cells into various parts of the brain has been disclosed. It has also been suggested to deliver therapeutic agents by infecting endothelial cells of blood vessels located in the brain with a viral vector, as a result of WO 98/32869 PCT/DK98/000372 intravascular administration of the vector to the host near the site of infection (WO 96/22112).

Alternatively, a broad range of viral vectors such as including adenovirus vectors (WO 95/26408), adeno-associated virus vectors (WO 95/34670), herpes virus 5 vectors (Glorioso et al. Sem. Virol. 1992 3 265-276), vaccinia virus vectors, and retroviral vectors, including systems based on HIV, have been suggested as delivery vehicles for therapeutic genes to the CNS. Such vectors can be administered to the CNS by the intravenous and the intracranial route, and by administration to the cerebrospinal fluid.

WO 95/09654 discloses a method for the treatment of adverse conditions of the CNS by administration of producer cells containing a retroviral vector to the cerebrospinal fluid. The producer cells produce retroviral particles which are capable of transducting cells present in the nervous system. Intracerebral implantation of encapsulated cells producing a therapeutic peptide has also been suggested.

Accordingly, in one aspect the present invention provides a recombinant expression vector comprising a gene encoding a therapeutically active Parkin D126 under transcriptional control of an ubiquitin promoter.

The recombinant expression vector of the invention may be any vector so suitable for transfecting Parkin D126. However, in a preferred embodiment, the recombinant expression vector is an eukaryotic expression vector or a recombinant viral expression vector.

A gene according to the present invention which codes for Parkin D126 can be transferred into a cell using a variety of means including calcium phosphate precipitation (Graham et al., Virol. 1973 52 456-467; Wigler et al., Cell 1979 777-785), electroporation (Neumann et al., EMBO J. 1982 841-845), microinjection (Graessmann et al., Meth. Enzymology 1983 101 482-492), by means of liposomes (Staubinger et al., Methods in Enzymology 1083 101 512-527), spheroblasts (Schaffner et al., Proc. Natl. Acad. Sci. USA, 1980 77 2163-2167), by means of recombinant viruses, or by other methods known to those skilled in the art.

Viral vectors provide an efficient means of transferring a gene into a cell both in vivo and in vitro. The efficiency of viral gene-transfer is due to the fact that transfer of DNA is an essential part of the natural life cycle of viruses and that DNA transfer is a receptor-mediated process. Several viral systems including retrovirus, adenovirus, adeno-associated virus, vaccinia virus and herpes virus have been developed as in vivo therapeutic gene transfer vectors for gene therapy of CNS disorders.

Viral vectors have also been used to transform various forms of cells, such as astrocytes, fibroblast cells and cerebral endothelial cells which were thereafter implanted into the CNS. WO 96/06942 further describes genetically altered T-cells which enter the CNS and may be used as gene transfer vehicles.

A recombinant viral expression vector of the present invention may be any viral expression vector suited for in vivo transfer and expression of genes. Preferred viral expression vectors include retroviral vectors, recombinant adenovirus vectors, recombinant adeno-associated virus vectors, vaccinia virus vectors and recombinant herpes virus vectors.

Retroviral vector systems used for the generation of recombinant retroviral particles consist of two components. The retroviral vector itself is a modified retrovirus (vector plasmid) in which the genes encoding the viral proteins (gag, pol and/or env) have been replaced by the nucleic acid sequences of the present invention, such as a gene coding for Parkin D126 and/or marker genes to be transferred to the target cell. Since the replacement of the genes encoding for the viral proteins effectively cripples the virus, it must be rescued by the second component in the system which provides the missing viral proteins to the modified retrovirus. The second component is a cell line that produces large quantities of the 25 viral proteins (e.g. gag, pol and/or env), but that however lacks the ability to produce replication competent virus. This cell line is known as the packaging cell line and consists of a cell line transfected with one or more plasmids carrying the genes (gag, pol and/or env) enabling the modified retroviral vector to be packaged. To generate the packaged vector, the vector plasmid is transfected into the packaging cell line. Under these conditions, the modified retroviral genome including the inserted Parkin D126 gene and any marker genes are transcribed from the vector plasmid and packaged into modified retroviral particles (recombinant viral particles). This recombinant virus is then used to infect target cells in vitro or in vivo.

The viral genome and a Parkin D126 gene and any carried marker genes become integrated into the target cell's DNA A cell infected with such a recombinant viral particle cannot produce a new vector virus, since no viral proteins are present in these cells. However, the DNA of the vector carrying the therapeutic and marker genes is integrated in the cell's DNA as a provirus and can now be expressed in the infected cell.

WO-A1-9607748 describes the principle and construction of a new type of retroviral vector. In the ProCon-vector plasmid the right-hand (3′) U3 region is altered, but the normal left-hand (5) U3 structure is maintained; the vector can be normally transcribed into RNA utilising the normal retroviral promoter located within the left-hand (5′) U3 region upon its introduction into packaging cells. However the generated RNA will only contain the altered right-hand (3′) U3 structure. In the infected target cell, after reverse transcription, this altered U3 structure will be present in both Long Terminal Repeat at either end of the retroviral structure.

If the altered region carries a polylinker instead of the U3 region (which contains the viral promoter) then any promoter can be inserted and this promoter is then utilised exclusively in the target cell for expression of linked sequences encoding therapeutic polypeptides.

DNA segments homologous to one or more cellular sequences can also be inserted into the polylinker for the purposes of gene targeting, by homologous recombination.

The retroviral vectors of the present invention need not be of the ProCon type, but can be any conventional retroviral vector carrying a Parkin D126 gene. Such vectors include Self-inactivating-Vectors (SIN) in which retroviral promoters are functionally inactivated in the target cell (WO-A1-94129437). Further modifications of these vectors include the insertion of promoter gene cassettes within the LTR region to create double copy vectors (WO-A1-89/11539). In both of these vectors the heterologous promoters are inserted either in the body of the vector, or in the LTR region that are directly linked to the therapeutic gene. The retroviral vectors of the invention are based preferably either on a BAG vector (Price, Turner J D, and Cepko C; Proc. Natl. Acad. Sci. USA 1987 84 156-160) or an LXSN vector (Miller A D, & Ross man G J; Biotechni4ues 1989 7 980-990).

Eukaryotic Expression Vectors

The recombinant eukaryotc expression vector of the invention may be any eukaryotic expression vector suited for transferring a gene to mammalian cells. Preferred eukaryotic expression vector of the invention are pTEJ-4, pTEJ-8, or pUbi1Z.

As eukaryotic expression vectors have to be propagated in bacteria prior to transfection of eukaryotic cells, the eukaryotic expression vector should contain sequences which facilitate the prokaryotic propagation along with eukaryotic transcription units.

Prokaryotic sequences include a bacterial resistance gene under the transcriptional control of the prokaryotic promoter e.g. EM7 and a bacterial origin of DNA replication. The eukaryotic transcription unit responsible for eukaryotic selection e.g. employs the SV 40 early promoter to drive the expression of a resistance gene and a polyadenylation signal.

The present expression vectors contain one of the following prokaryotic/eukaryotic selection markers: neomycin, hygromycin, pyromycin or zeocin resistance gene allowing the selection of bacterial clones under EM7 promoter and cellular clones under SV 40 early promoter. The second eukaryotic transcription unit contains a UbC promoter, a polyadenylation signal, and finally a polylinker for insertion of nucleotide sequences encoding the protein in question (as an example see pUbi1Z, of Example 2).

The neurological disorders contemplated according to the present invention are e.g. ischemic stroke; metabolic disease of the brain; axonal injury; spinal cord injury; Alzheimers disease; Lewy body variant of Alzheimer's disease; Multiple system atrophy; Amyothropic Lateral Sclerosis (ALS); Parkinson's disease; Huntington's disease; motor neurone disease; central nervous system infections; epilepsy; post polio syndrome; mucopolysaccharidoses (MPS), In particular MPS types I to VII; lipidosis, in particular Gaucher's disease; Lesch-Nyhan syndrome; X-linked ADL; metachromatic leukodystrophy; Krabbe's disease; Charcot-Marie-Tooth disease; Fragile X; epilepsy; Down's syndrome; phenylketonuria; or mental disorders.

The eukaryotic expression vectors and the recombinant retroviral vectors according to the invention may in addition to the therapeutic gene carry a gene encoding a marker. The marker may in particular be a protein comprising f3-galactosidase, alcohol dehydrogenase, luciferase, puromycin- and neomycin resistance proteins, hypoxanthine phosphoribosyl transferase (HPRT), hygromycin, secreted alkaline phosphatase, or green or blue fluorescent proteins (GFP).

Recipient Cells

In order to deliver the therapeutically active polypeptide to the CNS, the vector of the invention may be used to transduce cells to express the therapeutic Parkin D126 in vivo after being implanted into the CNS. If cells producing the therapeutically active Parkin D126 are generated by transfection with a plasmid vector, several recipient cells may be used, e.g. immortalised neural stem cells, immortalised cerebral endothelial cell, or other immortalised cells compatible with the CNS. If cells capable of secreting therapeutic virus particles, that are generated by transfection with a viral vector, are used for transplantation of the packaging cell, several cells may be used, e.g. immortalised neural stem cells, immortalised cerebral endothelial cell, or other immortalised cell compatible with CNS.

If therapeutic virus particles generated by transfecting a packaging cell with a viral vector should be used for direct injection into the CNS, a large number of human cells may be used, e.g. immortalised neural stem cells, immortalised cerebral endothelial cell, or fibroblast-like human cell, e.g. HEK 293 or HeLa.

The packaging cell line of the invention can be selected from an element of the group consisting of psi-2 (Mann R, Mulligan R C, & Buttimore D; Cell 1983 33 153159), psi-Crip (Danos O & Mulligan R C; Proc. Natl. Acad. Sci. USA 1988 85, 6460-25 6464), psi-AM (Cone R D, & Mulligan R C, Proc. Natl. Acad. Sci. USA, 1984 81 63496353), GP+E-86 (Markowitz D, Golf S, & Bank A; J. Virol. 1988 62 1120-1124), PA317 (Miller A D, & Buttimore C; Mol. Cell. Biol. 1986 6 2895-2902), GP+envAM-12 (Markowitz D, Goff S, & Bank A; VirOlOqy 1988 167 400-406), Boso 23, Bing (Pear W S, Nolan G P, Scott M L, & Buttimore D; Proc. Natl. Acad. Sci. USA, 1993 90 8392-30 8396) or FLYA13, FLYRDI 8 (Cosset F L, Takeuchl Y, Battini J L, Weiss R A, & Collins M K L; J. Virol. 1995 69 74309-7436), or of any of these transfected with recombinant constructs allowing expression of surface proteins from other enveloped viruses. Such pseudotyped retroviral particles are described in PCT/EP96/01348.

In a particular preferred embodiment, the packaging cell line is made from human cells, e.g. HT1080 cells (WO-A1-9621014), HEK 293, thereby allowing production of recombinant retrovirus that is capable of surviving inactivation by human serum.

In a particular embodiment, the invention is directed to the use of the recombinant expression vector of the invention for the manufacture of a pharmaceutical composition, useful for the treatment of a neurological disease or disorder. The pharmaceutical composition of the invention is preferably a composition suited for injection or implantation into the human brain. Such compositions may be provided in the form of vials of frozen cells, optionally provided in a freeze medium in suspension.

### EXAMPLES

### Example 1

Parkin Nucleotide and Amino Acid Sequence

LOCUS AB009973 2960 bp mRNA PRI14-APR-2000 DEFINITION Homo sapiens mRNA for Parkin, complete cds.ACCESSION AB009973 VERSION AB009973.1,GI:3063387

| Nucleotide sequence | |
|---|---|
| 1 | tccgggagga ttacccagga gaccgctggt gggaggcgcg gctggcgccg ctgcgcgcat |
| 61 | gggcctgttc ctggcccgca gccgccacct acccagtgac catgatagtg tttgtcaggt |
| 121 | tcaactccag ccatggtttc ccagtggagg tcgattctga caccagcatc ttccagctca |
| 181 | aggaggtggt tgctaagcga cagggggttc cggctgacca gttgcgtgtg attttcgcag |
| 241 | ggaaggagct gaggaatgac tggactgtgc agaattgtga cctggatcag cagagcattg |
| 301 | ttcacattgt gcagagaccg tggagaaaag gtcaagaaat gaatgcaact ggaggcgacg |
| 361 | accccagaaa cgcggcggga ggctgtgagc gggagcccca gagcttgact cgggtggacc |
| 421 | tcagcagctc agtcctccca ggagactctg tggggctggc tgtcattctg cacactgaca |
| 481 | gcaggaagga ctcaccacca gctggaagtc cagcaggtag atcaatctac aacagctttt |
| 541 | atgtgtattg caaaggcccc tgtcaaagag tgcagccggg aaaactcagg gtacagtgca |
| 601 | gcacctgcag gcaggcaacg ctcaccttga cccagggtcc atcttgctgg gatgatgttt |
| 661 | taattccaaa ccggatgagt ggtgaatgcc aatccccaca ctgccctggg actagtgcag |
| 721 | aatttttctt taaatgtgga gcacacccca cctctgacaa ggaaacacca gtagctttgc |
| 781 | acctgatcgc aacaaatagt cggaacatca cttgcattac gtgcacagac gtcaggagcc |
| 841 | ccgtcctggt tttccagtgc aactcccgcc acgtgatttg cttagactgt ttccacttat |
| 901 | actgtgtgac aagactcaat gatcggcagt ttgttcacga ccctcaactt ggctactccc |
| 961 | tgccttgtgt ggctggctgt cccaactcct tgattaaaga gctccatcac ttcaggattc |
| 1021 | tgggagaaga gcagtacaac cggtaccagc agtatggtgc agaggagtgt gtcctgcaga |
| 1081 | tggggggcgt gttatgcccc cgccctggct gtggagcggg gctgctgccg gagcctgacc |
| 1141 | agaggaaagt cacctgcgaa gggggcaatg gcctgggctg tgggtttgcc ttctgccggg |
| 1201 | aatgtaaaga agcgtaccat gaaggggagt gcagtgccgt atttgaagcc tcaggaacaa |
| 1261 | ctactcaggc ctacagagtc gatgaaagag ccgccgagca ggctcgttgg gaagcagcct |
| 1321 | ccaaagaaac catcaagaaa accaccaagc cctgtccccg ctgccatgta ccagtggaaa |
| 1381 | aaaatggagg ctgcatgcac atgaagtgtc cgcagcccca gtgcaggctc gagtggtgct |
| 1441 | ggaactgtgg ctgcgagtgg aaccgcgtct gcatggggga ccactggttc gacgtgtagc |
| 1501 | cagggcggcc gggcgcccca tcgccacatc ctgggggagc atacccagtg tctaccttca |
| 1561 | ttttctaatt ctcttttcaa acacacacac acacgcgcgc gcgcgcacac acactcttca |
| 1621 | agtttttttc aaagtccaac tacagccaaa ttgcagaaga aactcctgga tccctttcac |
| 1681 | tatgtccatg aaaaacagca gagtaaaatt acagaagaag ctcctgaatc cctttcagtt |
| 1741 | tgtccacaca agacagcaga gccatctgcg acaccaccaa caggcgttct cagcctccgg |
| 1801 | atgacacaaa taccagagca cagattcaag tgcaatccat gtatctgtat gggtcattct |
| 1861 | cacctgaatt cgagacaggc agaatcagta gctggagaga gagttctcac atttaatatc |
| 1921 | ctgcctttta ccttcagtaa acaccatgaa gatgccattg acaaggtgtt tctctgtaaa |
| 1981 | atgaactgca gtgggttctc caaactagat tcatggcttt aacagtaatg ttcttattta |
| 2041 | aattttcaga aagcatctat tcccaaagaa ccccaggcaa tagtcaaaaa catttgttta |
| 2101 | tccttaagaa ttccatctat ataaatcgca ttaatcgaaa taccaactat gtgtaaatca |
| 2161 | acttgtcaca aagtgagaaa ttatgaaagt taatttgaat gttgaatgtt tgaattacag |
| 2221 | ggaagaaatc aagttaatgt actttcattc cctttcatga tttgcaactt tagaaagaaa |
| 2281 | ttgtttttct gaaagtatca ccaaaaaatc tatagtttga ttctgagtat tcattttgca |
| 2341 | acttggagat tttgctaata catttggctc cactgtaaat ttaatagata aagtgcctat |
| 2401 | aaaggaaaca cgtttagaaa tgatttcaaa atgatattca atcttaacaa aagtgaacat |
| 2461 | tattaaatca gaatctttaa agaggagcct ttccagaact accaaaatga agacacgccc |
| 2521 | gactctctcc atcagaaggg tttatacccc tttggcacac cctctctgtc caatctgcaa |
| 2581 | gtcccaggga gctctgcata ccaggggttc cccaggagag accttctctt aggacagtaa |
| 2641 | actcactaga atattcctta tgttgacatg gattggattt cagttcaatc aaactttcag |
| 2701 | cttttttttc agccattcac aacacaatca aaagattaac aacactgcat gcggcaaacc |
| 2761 | gcatgctctt acccacacta cgcagaagag aaagtacaac cactatcttt tgttctacct |
| 2821 | gtattgtctg acttctcagg aagatcgtga acataactga gggcatgagt ctcactagca |
| 2881 | catggaggcc cttttggatt tagagactgt aaattattaa atcggcaaca gggcttctct |
| 2941 | ttttagatgt agcactgaaa |

Amino acid sequence
MIVFVRFNSSHGFPVEVDSDTSIFQLKEVVAKRQGVPADQLRVIFAGKELRNDWTVQNCDLDQQSIVHI
VQRPWRKGQEMNATGGDDPRNAAGGCEREPQSLTRVDLSSSVLPGDSVGLAVILHTDSRKDSPPAGSPA
GRSIYNSFYVYCKGPCQRVQPGKLRVQCSTCRQATLTLTQGPSCWDDVLIPNRMSGECQSPHCPGTSAE
FFFKCGAHPTSDKETPVALHLIATNSRNITCITCTDVRSPVLVFQCNSRHVICLDCFHLYCVTRLNDRQ
FVHDPQLGYSLPCVAGCPNSLIKELHHFRILGEEQYNRYQQYGAEECVLQMGGVLCPRPGCGAGLLPEP
DQRKVTCEGGNGLGCGFAFCRECKEAYHEGECSAVFEASGTTTQAYRVDERAAEQARWEAASKETIKKT
TKPCPRCHVPVEKNGGCMHMKCPQPQCRLEWCWNCGCEWNRVCMGDHWFDV

### Example 2

Parkin antibodies and their characterisation.

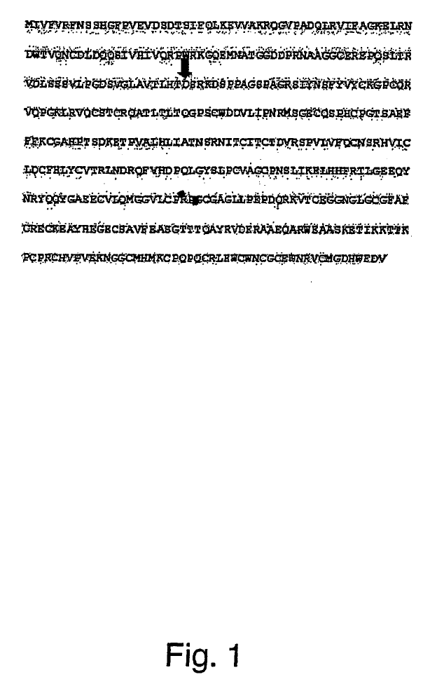

Localisation of the epitopes used for producing the antibodies PAR-N1, PAR-C1 and T160 (See panel A in FIG. 1).

The rabbit antibodies PAR-N1 and PAR-C1 were raised by immunising rabbits with the synthetic peptides Glu-Val-Asp-Ser-Asp-Thr-Ser-lle-Phe-Gln-Leu-Lys-Glu-Val-Val-Ala-Lys-Arg-Gln-Cys and Cys-Glu-Trp-Asn-Arg-Val-Cys-Met-Gly-Asp-His-trp-Phe-Asp-Val, respectively, both conjugated to keyhole limpet hemocyanine. Glu-Val-Asp-Ser-Asp-Thr-Ser-lle-Phe-Gln-Leu-Lys-Glu-Val-Val-Ala-Lys-Arg-Gln-Cys corresponds to the human Parkin amino acid sequence 16-34 with a Cys residue added to the C-terminus. Cys-Glu-Trp-Asn-Arg-Val-Cys-Met-Gly-Asp-His-trp-Phe-Asp-Val corresponds to the human Parkin amino acid sequence 451-465 with a Cys residue added to the N-terminus. The antibody T160 was raised by immunisation of rabbits with a partially purified recombinant human Parkin protein where the N-terminus was extended with 6 histidine residues. All antibodies were used as sera.

The chinese hamster ovary (CHO) cell line K1 and the human neuroblastoma cell line SH-SY5Y were stably transfected with an expression vector that lead to expression of human Parkin protein in the cells. The eukaryotic expression vector was generated by amplifying cDNA coding human Parkin with primer I and II by polymerase chain reaction and ligated into the pcDNA3.1/zeo(−) vector.

| | |
|---|---|
| Primer I; | CACGGATTCATGATAGTGTTTGTCAGG, |
| Primer II; | GAGCTAGCCACCATGATAGTGTTTGTCAGG. |

The parental cell lines and the Parkin expressing cell lines were extracted in 8 M urea, 1% Triton X 100.20 microgram protein was supplemented to 2% sodium dodecylsulphate, 20 mM dithioerythreitol, 20 mM Tris, pH 6.5, 20% glycerol, heated to 95° C. for 5 min, whereafter the mixture was resolved by 10-20% gradient sodium dodecylsulphate polyacrylamide gelelectrophoresis. The proteins in the gel were electroblotted onto a nitrocellulose membrane and non-specific protein binding was blocked by incubating the membrane in block solution (20 mM Tris, pH 7.4, 130 mM NaCl, 0.5% Triton X100, 5% skimmed milk powder) for 2 hours. The membrane was subsequently incubated with primary antibody (PAR-N1, PAR-C1 and T160 diluted 1/500 in block solution) for 2 hours, rinsed in 20 mM Tris, pH 7.4, 130 mM NaCl, 0.5% Triton X100 and incubated with a second antibody (horse radish peroxidase conjugated swine anti rabbit serum) for 1 hour and rinsed. The bound antibody was then visualised by chemilumenescense according to the manufacturers recommendation (ECL, Amersham Pharmacia Biotech). FIG. 2 panel B, lanes 1 and 5 represent the non-transfected CHO and SH-SY5Y cell lines, respectively. FIG. 2, panel B, lanes 24 represents Parkin expressing CHO cells K3 and lane 6 represents the Parkin expressing SH-SY5Y cell Park 8. Lanes 1, 2, 5 and 6 were incubated with T160, lane 3 with PAR-Ni and lane 4 with PAR-C1. The molecular size marker is indicated to the left in kDa.

Immunoprecipitation of Metabolically Labeled Recombinant Human Parkin

The Parkin expressing K3 CHO cell lines were incubated with 35S-labelled methionine (200 microCi/ml) in a methionine free medium for 4 hours whereafter the cells were lysed in 8 M urea. Insoluble material was removed by centrifugation at 13,000×g for 15 min. The soluble cell extract was diluted 20 fold in 130 mM NaCl, 20 mM Tris, pH 7.4, 1 mM EDTA supplemented with the proteinase inhibitor mixture (Complete, Roche) and incubated with 2 microliter of one of the antisera PAR-N1, PAR-C1, T160 or a non-immune serum for 16 h at 4° C. to facilitate binding to the labeled Parkin. The incubate was supplemented With 40 microliter protein A coupled to Sepharose (Amersham-Pharmacia Biotech) for 1 h at 4° C., whereafter the protein A-Sepharose with the bound antibodies was collected by centrifugation followed by 5×wash in 10 ml 130 mM NaCl, 20 mM Tris, pH 7.4, 1 mM EDTA, 0.05% Triton X100 was supplemented with the proteinase inhibitor mixture (Complete, Roche). The washed Protein-A Sepharose was added to 40 microliter 2% sodium dodecylsulphate, 20 mM dithioerytreitol, 20 mM Tris, pH 6.5, 20% glycerol and heated to 95° C. for 5 min, whereafter the mixture was resolved by 10-20% gradient sodium dodecylsulphate polyacrylamide gelelectrophoresis. The gel was dried and the radioactivity in the gel was visualised by autoradiography. Lanes 1-4 in FIG. 2, panel C shows the radioactivity precipitated by the antibodies PAR-N1, PAR-C1, T160 and the non-immune IgG, respectively. The position of the 52 kDa Parkin band is shown by an arrow to the left.

### Example 3

Parkin is Cleaved During Cellular Apoptosis

The stably transfected Parkin expressing cell lines, K3-CHO (FIG. 3, lanes 1-5) and Park8-SH-SY5Y (FIG. 3, lanes 6, 7) were extracted and prepared for immunoblotting as described in example 2. The cells were cultured in the absence (FIG. 3, lanes 1) and presence of the inducers of apoptosis; okadaic acid (200 nM for 16 hours, lanes 2, 5-7), staurosporin (10 microM/ml for 16 hours, lane 3) and campthotecin (5 microgram/ml for 30 hours, lane 4). FIG. 3, lanes 1-4, 6 were probed with T160, lane 5 with PAR-N1 and lane 7 with PAR-C1 antibodies. The molecular weight is indicated to the left for Parkin (51 kDa) and the Parkin peptides (38 kDa and 11,5 kDa, respectively). The induction of apoptosis was verified biochemically by testing the binding of the same membranes with the mouse monoclonal poly-ADP-ribose (PARP) antibody 66401A (Pharmigen). This antibody recognised only the full length 116 kDa PARP protein in the control cells whereas the caspase 3 generated 86 kDa fragment was generated in the cells treated with the inducers of apotosis. The generation of the 86 kDa fragment is a recognised marker for apoptosis-associated proteolysis.

### Example 4

Caspase Inhibitors Inhibit the Apoptosis-associated Parkin Cleavage

K3-CHO cells were treated with 200 nM okadaic acid for 16 h prior to extraction and processing for T160 immunoblot as described in example 2. FIG. 4, lane 1 demonstrates the non-cleaved parkin in the untreated cells, whereas the parkin in the okadaic-treated cells is demonstrated in lanes 2-4. FIG. 4, lanes 3 and 4 represent cells supplemented with 10 microM of the caspase inhibitors WAD-chloromethylketone and DEVD-chloromethylketone, respectively. The arrows to the left demonstrate the position of the 51 kDa Parkin and 38 kDa Parkin fragment. Evidently, the inhibitors abrogate the cleavage. Similar data was obtained in the Park8 SH-SY5Y cells.

### Example 5

Cell Lines Expressing D126E Mutant Parkin

The cDNA encoding Parkin was kindly provided by Dr. Mizuno, Juntendo University School of Medicine, Japan. Parkin cDNA was PCR amplified using the primers:

5′-GAGCTAGCCACCATGATAGTGTTTGTCAGG-3′ and
5′-GTGAATTCCTACACGTCGAACCAG-3′.

The amplified Parkin cDNA was cloned into a Nhe I and EcoR I site of pcDNA3.1/Zeo(−) vector and the parkin cDNA sequence was checked by sequencing. To prepare the D126E mutant, a site-directed mutagenesis was performed by using the Quickchange Mutagenesis Kit (Stratagene) according to the manufacturer's instructions.

Complementary strands of the oligonucleotide: 5′-GTCATTCTGCACACTGAAAGCAGGAAGGACTCACC-3′ was used for preparing the D126E mutated construct. This Vector was then transfected into SH-SY5Y or CHO cells using FUGENE6 (Boehringer Mannheim) and stable transfected cell lines were selected using 25 microgram Zeocin/ml medium (SH-SY5Y cells) or 300 microgram Zeocin/ml medium (CHO cells).

### Example 6

Site Directed Mutagenesis of Aspartate 126 in Parkin to a Glutamate Residue Reduce the Apoptosis Associated Cleavage of Parkin

Caspases are proteinases that always cleave after an Asp residue. Cell lines were generated where Asp residues were changed to Glu residues to test if this amino acid position might represent the caspase cleavage sites. The Parkin expression vector used for generating the Parkin expressing K3 CHO and Park8 SH-SY5Y cell lines was changed by PCR mediated site directed mutagenesis so the nucleotide No.479 was changed from a cytosine to an adenine (ParkinD126E). The cDNA encoding Parkin was kindly provided by Dr. Mizuno, Juntendo University School of Medicine, Japan. The Parkin cDNA was PCR amplified using the primers:

5′-GAGCTAGCCACCATGATAGTGTTTGTCAGG-3′ and
5′-GTGAATTCCTACACGTCGAACCAG-3′.

The amplified Parkin cDNA was cloned into the Nhe I and EcoR I site of the pcDNA3.1/Zeo(−) vector and the Parkin cDNA sequence was checked by sequencing. To prepare the D126E mutant, the inventors performed site-directed mutagenesis using the Quickchange Mutagenesis Kit (Stratagene) according to the manufactures instructions. Complementary strands of the oligonucleotide: 5′-GTCATTCTGCACACTGAMGCAGGMGGACTCACC-3′ were used for preparing the D126E mutated construct. Parkin vectors containing Parkin mutated for D130E and DD86, 87EE were constructed by analogous techniques. This vector was then transfected into SH-SYSY or CHO cells using FUGENE6 (Boehringer Mannheim) and stably transfected cell lines were selected using 25 microgram Zeocinl ml medium (SH-SY5Y cells) or 300 microgram Zeocin/ml medium (CHO cells). The sequences of the vectors were verified by DNA sequencing. The expression vectors were used to generate the stably transfected DD86, 87EE, D126E and D130E CHO and SH-SY5Y cell lines expressing Parkin protein with the indicated substitutions of amino acids. These cells and the cell lines expressing the normal Parkin were treated with 200 nanoM okadaic acid for 16 h and extracted for T160 immunoblotting. The CHO cell lines are represented in lanes FIG. 5, 1-6 and the SH-SY5Y cell lines in lanes 7-12. Wild type Parkin is represented in lanes 1, 2, 7, 8; D126E in lanes 3, 4, 9, 10; D130E in lanes 5, 6, 11, 12. Lanes 1, 3, 5, 7, 9, 11 represent untreated cells and lanes 2, 4, 6, 8, 10, 12 represent okadaic treated cells. The positions of the 51 kDa and 38 kDa Parkin proteins are indicated by the upper and lower left arrows. The experiments demonstrated that the D126E point mutation abrogated the cleavage whereas the D130E and the double mutant DD86, 87EE had no significant effect.

### Example 7

Demonstration of a Cytoprotective Effect of Inhibiting D126-cleavage in Parkin

Parkin expression protects toward unfolded protein stress as demonstrated by Imai et al., (Imai et al., (2000) J Biol Chem Vol.275, No.46, Nov 17, pp. 35661-35664) The inventors adopted their assay demonstrated in FIG. 4A of (Imai et al., (2000) J Biol Chem Vol.275, No.46, Nov 17, pp. 35661-35664), where Parkin expression is proven to give an approximately 50% protection against cell death induced by 3 mM mercaptoethanol.

Mercaptoethanol is a known inducer of unfolded protein stress by inhibiting the normal formation of disulfide bonds in the endoplasmic reticulum.

CHO and SH-SY5Y cell lines were cultured expressing wild type Parkin, D126E Parkin and an empty vector with 3 mM mercaptoethanol for 24h. They were stained with DNA binding fluorescent dye DAPI and fixed in ice cold methanol prior to drying. Prior to this analysis it was demonstrated that mercaptoethanol induced cell death is followed by Parkin cleavage after D126 by T160 immunoblotting as described in example 3. The percentage of dead cells were then counted on an inverted flourescense microscope as the percentage of nuclei exhibiting condensed brightly fluorescent DNA as compared to the unaffected evenly fluorescent normally appearing nuclei. The cytoprotective effect of inhibiting the D126 cleavage was demonstrated by the enhanced survival of cells expressing the D126E Parkin as compared to wild type Parkin.

### Example 8

Demonstration of Specific Parkin Cleavage After D126 by Antibody Recognition.

Proteolytic cleavage is known to liberate neoepitopes that enable the specific detection of the cleaved peptide species as exemplified by the many antibodies against activated caspases and the recently demonstrated caspase cleavage of Alzheimer's amyloid precursor protein (Gervais et al.,1999, Cell, 97,395-406).

To produce antibodies that specifically recognise a Parkin, which is cleaved after D126, a modified strategy of the one used in (Gervais et al. 1999 Cell Volume 97, 396405) was employed as described in detail in the following.

The Parkin sequence 117-139 with the cleavage site marked by a horisontal line: VGLAVILHTD-SRKDSPPAGSPAG was used to generate 2 synthetic peptides.

Peptide 1: SRKDSPPAGSC corresponding to Parkin127-136 with a cysteine residue added to the C-terminus.

Peptide 2: VILHTDSRKDSPPAGSPC corresponding to Parkin121-137 with a cysteine residue added to the C-terminus.

Peptide 1 was coupled to Keyhole limpet hemocyanine and used to immunise rabbits to generate antibodies that recognise Parkin epitopes just C-terminal to the cleavage site D126.

These antisera were subsequently affinity purified on two CNBr-activated Sepharose columns with immobilised peptide 1 and peptide 2, respectively. First the sera was depleted for antibodies that recognise uncleaved Parkin by passing it through the peptide 2 column. The antibodies that bound the immunising peptide were then captured by binding to the peptide 1 column whereafter they were eluted by 0.2 M acetic acid. The eluted and neutralised antibodies were finally passed through the peptide 2 column to remove any remaining antibodies that bound the uncleaved peptide. These final antibodies in the flow through were designated park126CLE.

The specificity of park126CLE was verified by demonstrating its binding to Parkin cleavage products generated in apoptotic cell lines expressing wild type Parkin but not in the D126E mutant. The antibodies will subsequently be used to monitor the generation of the D126-cleaved Parkin in cell lines by immunoflourescense microscopy and immunoblofting on cell extracts. Importantly, the antibody will be used to demonstrate the presence of D126-cleaved Parkin species in human brain tissue and extracts by immunohistochemistry and immunoblotting. The tissues examined will represent normal control tissue and tissue affected by Parkinson's disease, Alzheimer's disease and other neurodegenerative diseases.

The antibody will finally be used as a tool for the screening of small molecule libraries for inhibitors of D126-parkin cleavage in various cellular and isolated high throughput screeningassays.

claims 1-88. (canceled)

89. An isolated nucleic acid sequence which is at least 70% identical to the nucleic acid sequence shown in SEQ ID NO: 3, coding for a polypeptide functionally homologous to a mutated Parkin polypeptide, the amino acid sequence of said polypeptide differing from the wild type amino acid sequence such as shown in SEQ ID NO: 2 at least in including a mutation at Asp 126 or in the close vicinity thereof.

90. The isolated nucleic acid sequence according to claim 89, wherein said encoded polypeptide is characterised by
a) at least including said mutation at Asp 126 or in the close vicinity thereof, and
b) having an amino acid sequence that is at least 70% identical to an amino acid sequence shown in SEQ ID NO: 4.

91. An isolated nucleic acid sequence, coding for an amino acid sequence as shown in SEQ ID NO: 4, including a point mutation at Asp 126.

92. An isolated nucleic acid sequence complementary to a nucleic acid sequence according to claim 89.

93. The isolated nucleic acid sequence according to claim 89, coding for a Parkin polypeptide in which cleavage at Asp 126 or in the close vicinity thereof is altered.

94. The isolated nucleic acid sequence according to claim 89, coding for a Parkin polypeptide in which proteolytic processing at Asp 126 or in the close vicinity thereof by a protease is prevented.

95. The isolated nucleic acid according to claim 94, wherein said protease is a cysteine proteinase that cleaves a substrate after an aspartic acid residue.

96. The isolated nucleic acid sequence according to claim 95, wherein said protease is a caspase.

97. A purified Parkin polypeptide having an amino acid sequence that is at least 70% identical to the amino acid sequence shown in SEQ ID NO: 4, the amino acid sequence differing from the wild type amino acid sequence shown in SEQ ID NO 2 at least in including a point mutation at Asp 126 or the close vicinity thereof.

98. The purified polypeptide according to claim 97, which is functionally homologous to a mutated Parkin polypeptide.

99. The purified polypeptide according to claim 97, wherein said point mutation at Asp 126 or the close vicinity thereof alters a potential cleavage site of said polypeptide.

100. The purified polypeptide according to claim 97, wherein said point mutation at Asp 126 or the close vicinity thereof prevents proteolytic processing of said polypeptide in a mammalian cell.

101. The purified polypeptide according to claim 98, wherein said point mutation at Asp 126 or the close vicinity thereof prevents proteolytic processing of Parkin by a protease.

102. The purified polypeptide according to claim 101, wherein said protease is a cysteine proteinase that cleaves a substrate after an aspartic acid recidue.

103. The purified polypeptide according to claim 98, wherein said point mutation at Asp 126 or close vicinity thereof prevents proteolytic processing of Parkin by a caspase.

104. A vector comprising a nucleic acid sequence corresponding to or being complementary to a nucleic acid sequence, which is at least 70% identical to the nucleic acid sequence shown in SEQ ID NO: 3, coding for a polypeptide functionally homologous to a mutated Parkin polypeptide, the amino acid sequence of said polypeptide differing from the wild type amino acid sequence shown in SEQ ID NO: 2 at least in including a mutation at Asp 126 or in the close vicinity thereof.

105. The vector according to claim 104, comprising a nucleic acid sequence coding for a polypeptide having an amino acid sequence that is at least 70% identical to the amino acid sequence shown in SEQ ID NO: 4, the amino acid sequence differing from the wild type amino acid sequence shown in SEQ ID NO 2 at least in including a point mutation at Asp 126 or the close vicinity thereof.

106. A host cell, a host cell line, or a primary tissue culture host transformed with a vector according to claim 104.

107. The host cell, a host cell line, or a primary tissue culture host according to claim 106, wherein the host is a eukaryotic cell.

108. A host cell according to claim 106, the host cell being a mammalian stem cell, or stem cell line, which expresses a transgenic Parkin, wherein said transgenic Parkin at least differs from the wild type amino acid sequence by being more resistant to proteolytic processing at a potential cleavage site at Asp 126 or the close vicinity thereof than wild type Parkin.

109. An antibody that selectively binds to a polypeptide fragment of Parkin, wherein said fragment is the result of cleavage of Parkin at 126 or the close vicinity thereof.

110. The antibody of claim 109, selectively binding to a region of said cleaved Parkin polypeptide fragment, located within 50 amino acids from Asp126 on either side thereof.

111. The antibody of claim 109, wherein said polypeptide fragment as a result of the cleavage of Parkin at Asp 126 has a changed conformational structure compared to the original conformational structure of the corresponding uncleaved Parkin, and wherein said antibody binds to said polypeptide fragment having a changed conformational structure.

112. The antibody according to claim 109 that binds a peptide corresponding to SEQ ID NO: 5.

113. The antibody according to claim 110, which does not bind a peptide corresponding to SEQ ID NO: 6.

114. A method for altering and/or inhibiting the proteolytic processing of Parkin at the potential cleavage site of said Parkin at Asp 126 or in the close vicinity thereof in an amino acid sequence shown in SEQ ID NO: 2, characterised by
i) providing a small organic compound that specifically binds to said site, or in close proximity thereof, thereby altering the accessibility of the cleavage site; and/or
ii) providing a small organic compound and/or small peptide or peptide fragment which acts as a protease inhibitor, as a ligand for a protease and/or as an activator of a protease inhibitor; and/or
iii) providing an activator of a kinase or an activator of a kinase activator, thereby increasing the level of phosphorylation of Parkin; and/or
iv) providing an activator of a phosphatase or an activator of a phosphatase activator, thereby decreasing the level of phosphorylation of Parkin.

115. The method according to claim 114, wherein said small peptide is a tetrapeptide consisting of Leu-His-Thr-Asp such as shown in SEQ ID NO: 7.

116. The method according to claim 114, wherein proteolytic processing of Parkin by a protease is at least partially prevented in a mammalian cell.

117. The method according to claim 116, wherein said protease is a caspase.

118. A method of using the antibody of claim 111, said method comprising detecting the occurrence of proteolytic processing of Parkin at the potential cleavage site of Parkin at Asp 126 or the close vicinity thereof in an amino acid sequence shown in SEQ ID NO: 2 in a sample of a mammal, in a mammalian cell, cell culture and/or primary tissue culture, said method comprising contacting said sample with said antibody.

119. The method according to claim 118, wherein said sample is selected from the list comprising blood, urine, faeces, spinal fluid, lymph, sputum, hair, nail, muscle biopsy, skin biopsi, brain tissue, or any solid tissue sample.

120. The method according to claim 118 for monitoring a potential disposition for a neurodegenerative disease in a mammal, wherein said sample said mammalian cell, cell culture and/or primary tissue culture; is exposed to one or more stress factors and the rate of proteolytically processed Parkin at Asp 126 or the close vicinity thereof in the Parkin amino acid sequence such as shown in SEQ ID NO: 2 is measured and compared to the amount of proteolytically processed Parkin in an untreated sample of same origin.

121. The method according to claim 118, for diagnosing PD and/or a potential disposition for PD in a mammal.

122. The method according to claim 121, wherein the mammal is a human.

123. The method according to claim 118 for testing a potential pharmaceutical substance for treating and/or preventing PD, characterised by applying said substance or a formulation comprising said substance to a neuronal cell, cell culture and/or to a primary culture of neuronal cells or another, non-neuronal cell or cell culture or primary cell culture and measuring the rate of proteolytically processed Parkin at Asp 126 or the close vicinity thereof in the Parkin amino acid sequence such as shown in SEQ ID NO: 2, and comparing said processing to the amount of proteolytically processed Parkin in an cell, cell culture and/or a primary culture of cells of same origin, or in cells expressing Parkin amino acid sequence such as shown in SEQ ID NO: 2.

124. The method of claim 123, wherein said cell is from a patient suffering from a neurodegenerative disease.

125. A method of using the antibody of claim 111, comprising testing a potential pharmaceutical substance for treating and/or preventing PD in a cell-free system, characterised by applying said substance or a formulation comprising said substance to a cell free system and detecting the occurrence of proteolytic processing of Parkin at the potential cleavage site of said Parkin at Asp 126 or the close vicinity thereof in an amino acid sequence such as shown in SEQ ID NO: 2 with said antibody.

126. The method according to claim 125 for testing a potential pharmaceutical substance for treating and/or preventing PD.

127. A method for preventing or treating neuronal cell degeneration in the CNS of a mammal, characterised by preventing and/or altering proteolytic processing of Parkin at Asp 126 or the close vicinity thereof in a Parkin amino acid sequence shown in SEQ ID NO: 2.

128. The method according to claim 127, wherein the mammal is a human in need thereof.

129. The method of claim 127, comprising treating and/or preventing a neurological disorder in a patient in need thereof, characterised by transplanting into the CNS of said patient a mammalian cell, which cell expresses a transgenic Parkin, wherein said transgenic Parkin at least differs from the wild type amino acid sequence by being more resistant to proteolytic processing at a potential cleavage site at Asp 126 or the close vicinity thereof than wild type Parkin.

130. The method of claim 127, for treating and/or preventing a neurological disorder in a patient, characterised by injecting or transplanting into the CNS of a patient in need thereof a vector for transforming neuronal cells, said vector being a viral vector, and said vector comprising a nucleic acid sequence corresponding to or being complementary to a nucleic acid sequence, which is at least 70% identical to the nucleic acid sequence shown in SEQ ID NO: 3, coding for a polypeptide functionally homologous to a mutated Parkin polypeptide, the amino acid sequence of said polypeptide differing from the wild type amino acid sequence such as shown in SEQ ID NO: 2 at least in including a mutation at Asp 126 or in the close vicinity thereof.

131. A pharmaceutical composition for treating and/or preventing a neurological disorder in a mammal comprising a suitable pharmaceutical carrier and:
i) a small organic compound that specifically binds to a potential cleavage site of Parkin at Asp 126 in an amino acid sequence shown in SEQ ID NO: 2, or in close proximity thereof; or
ii) a small organic compound that acts as a proteinase inhibitor and/or as a ligand for a protease and/or as an activator of a protease inhibitor and which alters a potential cleavage of Parkin at Asp 126 in an amino acid sequence shown in SEQ ID NO: 2, or in close proximity thereof, or
iii) a small peptide or peptide fragment which acts as a protease inhibitor, as a ligand for a protease and/or as an activator of a protease inhibitor and which alters a potential cleavage of Parkin at Asp 126 in an amino acid sequence shown in SEQ ID NO: 2, or in close proximity thereof, or
iv) a small organic compound that acts as an activator of a kinase or as an activator of a kinase activator and which alters a potential cleavage of Parkin at Asp 126 in an amino acid sequence shown in SEQ ID NO: 2, or in close proximity thereof; or
v) a small organic compound that acts as an activator of a phosphatase or as an activator of a phosphatase activator and which alters a potential cleavage of Parkin at Asp 126 in an amino acid sequence shown in SEQ ID NO: 2, or in close proximity thereof.


SEQUENCE LISTING (7 sequences)

SEQ ID NO: 1 - DNA, 2960 bp, Homo sapiens
  mRNA (1)..(2960)
tccgggaggattacccaggagaccgctggtgggaggcgcggctggcgccgctgcgcgcatgggcctgttcctggcccgcagccgccacctacccagtgaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacaccagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatcgaaataccaactatgtgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaa

SEQ ID NO: 2 - protein, 465 aa, Homo sapiens
  mat_peptide (1)..(): Native Parkin
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 3 - DNA, 2960 bp, Homo sapiens
  mRNA (1)..(2960)
tccgggaggattacccaggagaccgctggtgggaggcgcggctggcgccgctgcgcgcatgggcctgttcctggcccgcagccgccacctacccagtgaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgaaagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacaccagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatcgaaataccaactatgtgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaa

SEQ ID NO: 4 - protein, 465 aa, Homo sapiens
  mat_peptide (1)..(): Parkin with a D126E mutation
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTESRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 5 - protein, 11 aa, Homo sapiens
  MISC_FEATURE (1)..(11): Parkin 127-136 with C added to C-terminal end; for use as immnogen
SRKDSPPAGSC

SEQ ID NO: 6 - protein, 18 aa, Homo sapiens
  MISC_FEATURE (1)..(18): Parkin 121-137 with C added to C-terminal end; for use ad immunogen
VILHTDSRKDSPPAGSPC

SEQ ID NO: 7 - protein, 4 aa, Homo sapiens
  MISC_FEATURE (1)..(4): Inhibitor of Parkin cleavage amino acid sequence
LHTD